(19) United States
(12) Patent Application Publication
(10) Pub. No.: US 2026/0151234 A1
(43) Pub. Date: Jun. 4, 2026

(54) COMPUTERIZED METHOD OF MAKING A PATIENT-SPECIFIC ORTHOPEDIC RECONSTRUCTION

(71) Applicant: Blue Sky Technologies, LLC, Louisville, KY (US)

(72) Inventor: Frank Castro, Louisville, KY (US)

(21) Appl. No.: 19/449,621

(22) Filed: Jan. 15, 2026

Related U.S. Application Data
(63) Continuation-in-part of application No. 18/088,531, filed on Dec. 24, 2022, now Pat. No. 12,564,430.
(60) Provisional application No. 63/310,189, filed on Feb. 15, 2022.
(60) Provisional application No. 63/317,041, filed on Mar. 6, 2022.

(51) Int. Cl. A61F 2/30; A61F 2/44

(52) U.S. Cl. CPC A61F 2/30942; A61F 2/30767; A61F 2/4455; A61F 2002/30011; A61F 2002/30948; A61F 2002/30952

(57) ABSTRACT

A computerized system and method (123A-C) for the automated design and additive manufacturing of patient-specific orthopedic reconstructions (600R). The method integrates anatomically tagged medical scans, oriented relative to X, Y, and Z axes, with machine-learning models trained on aggregate historical biomechanical outcomes to generate optimized implant geometries. The system calculates an Osseointegration Probability Score (OPS) and a Comprehensive Prognostic Success Score (CPSS) to dynamically modify internal porous lattice densities, minimizing stress shielding while maximizing load-bearing capacity. The manufacturing process simultaneously generates internal longitudinal conduits for post-operative vascularization and computationally optimized structural anchors (bulges, serrations, and wings) for precise anatomical fixation. By linking real-time biomechanical simulation with prognostic clinical data, the invention provides a high-fidelity manufacturing blueprint that improves long-term implant stability and reduces periprosthetic complications.

### PRIORITY

This Application is a continuation-in-part of U.S. patent application Ser. No. 18/088,531, filed Dec. 24, 2022, which claims the benefit of U.S. Provisional Application No. 63/310,189, filed Feb. 15, 2022, and U.S. Provisional Application No. 63/317,041, filed Mar. 3, 2022. The entire contents of each of the above-identified applications are incorporated herein by reference.

### CROSS-REFERENCE TO RELATED APPLICATIONS

This application is a continuation-in-part of U.S. patent application Ser. No. 18/088,531, filed on Dec. 24, 2022, which published as U.S. Patent Application Publication No. 20230255690A1. The entire disclosure of the aforementioned US Published Patent Application 20230255690A1 is incorporated by reference herein in its entirety for all purposes as if fully set forth herein.

### BACKGROUND OF THE INVENTION

### Field of the Invention

Among other things, the present invention uses a process to make a patient-specific implant for one or more target zones within bone. Fixation or other types of implants or reconstructions can be manufactured by the computerized process (123) or the computerized methods (123A, 123B, 123C).

### Description of the Previous Art

References that may indicate a state-of-the-art for the current invention include:
1) US Published Patent Application 20150223939—Miles et al. discloses a method incorporating computer-implemented steps, a computing device and a computer readable storage medium for developing manufacturing parameters for manufacturing an orthopaedic implant; 2) US Published Patent Application 20140081400—Azernikov et al discloses semi-automatic customization of plates for internal fracture fixation; 3) US Published Patent Application 20180353299—Wei discloses 3D printing of mesh implants for bone delivery; 4) US Published Patent Application 20200129296—Chary et al. discloses implantable device for temporomandibular joint and method of production thereof; 5) U.S. Pat. No. 6,532,299—Sachdeva et al. discloses a system and method for mapping a surface; 6) US Published Patent Application 20080275558—Clifford et al. discloses extra-articular implantable mechanical energy absorbing systems and implantation method; 7) US Published Patent Application 20190321193—Casey et al. discloses systems and methods for orthopedic implantation fixation; 8) U.S. Pat. No. 10,799,295—Tjon discloses computer-aided design and preparation of bone graft; 9) US Published Patent Application 20040015327—Sachdeva et al discloses a unified workstation for virtual craniofacial diagnosis, treatment planning and therapeutics; 10) US Published Patent Application 20200170802—Casey discloses systems and methods for orthopedic implants; 11) US Published Patent Application 20190167435—Cordonnier discloses systems and methods for multi-planar orthopedic alignment; 12) US Published Patent Application 20220039965—Casey et al. discloses patient-specific artificial discs, implants and associated systems and methods; 13) US Published Patent Application 20180271661—Kamer et al. discloses method for manufacturing an auxiliary device suitable for the manufacture of a patient customized implant; 14) US Published Patent Application 20210053291—Bouvier et al. discloses method for manufacturing a complex substitute object from a real object; 15) US Published Patent Application 20190231436A1—Panse, et al discloses an anatomical model for position planning and tool guidance of a medical tool; 16) US Published Patent Application 20230277246A1—Casey et al. discloses a patient-specific implant design and manufacturing system with a digital filing cabinet manager; 17) US Published Patent Application 20230081437A1—Russell discloses a patient 3-D scanning and methods for optimizing port placement; 18) U.S. Pat. No. 12,220,174B2—Khan et al disclose an implant fit analysis; 19) U.S. Pat. No. 10,032,269B2—Lelong et al. discloses a method of determining an indicator for the stability of a bone implant; 20) U.S. Pat. No. 11,158,415B2—Daley discloses a surgical procedure planning system with multiple feedback loops; 21) US Published Patent Application 20170231771A1—Piron et al discloses feedback for providing artificial bone flap; 22) U.S. Pat. No. 6,532,299—Fairhall discloses system and method for mapping a surface; 23) U.S. Pat. No. 10,799,295—Tjon discloses computer-aided design and preparation of bone graft; 24) U.S. Pat. No. 7,993,347—Michlelson discloses a guard for use in performing human interbody spinal surgery; 25) U.S. Pat. No. 8,012,186—Pham et al discloses a uniplanar screw; 26) U.S. Pat. No. 8,764,804—Rezach discloses a bone fastener and methods of use; and 27) U.S. Pat. No. 9,826,986—Donner et al discloses systems for and methods of preparing a sacroiliac joint for fusion. While the prior art, such as Casey et al. (US 20230288246), discloses the general use of scans to construct three-dimensional models and the use of encrypted healthcare formatting, it fails to address the critical gap between a geometric fit and a biological success. Current state-of-the-art implants often fail due to ‘stress shielding’—where a metal implant is too rigid for the surrounding bone—or poor osseointegration, which the prior art cannot predict or mitigate.

The present invention solves this long-felt need by moving beyond a “passive” model to an active, predictive ecosystem. By utilizing the Osseointegration Probability Score (OPS) and the Comprehensive Prognostic Success Score (CPSS), the computerized method (123A-C) identifies the optimal non-uniform lattice density and material composition (e.g., graphene or bioactive polymers) for each unique patient. This ensures that the reconstruction is not just a physical match, but a functional and biological extension of the patient's natural anatomy, specifically optimized for long-term survival and quality of life.

### SUMMARY OF THE INVENTION

Process (123) and methods (123A, 123B, 123C) meet the long felt but unfulfilled need of manufacturing a patient-specific implants or reconstructions tailored specifically for the dimensions of the patient's target zone with consideration given to the physiological conditions in the target zone and the medical conditions of the patient.

Along with providing a patient-specific implant, process (123) can generate a model of the target zone, reports for the benefit of patients, medical personnel, insurers, hospitals, and manufacturers. One report can detail the advantages and disadvantages of several patient-specific implant options during the pre-operative period. Another report can provide technical guidance during the surgical procedure. And other reports can suggest rehabilitation protocols during the post-operative period. Software associated with the current process of making a patient-specific implant can utilize cumulative data from all previously implanted patient-specific devices to increase the probability of a successful patient outcome for all subsequent patient-specific device implantations.

Computerized methods (123A, 123B, 123C) can manufacture implants using methodology similar to process (123). However, computerized methods of making a patient-specific orthopedic reconstruction (123A, 123B, 123C) can manufacture bone implants (600R), bone replacement segments (600R), complete bone replacements (600R) and functioning bionics (600R).

Patient-specific orthopedic three-dimensional reconstructions (600R) are improvements to current state-of-the-art and novel devices utilized for bone surgeries or replacements. Biomechanical and biological stabilization provided by the use of patient-specific orthopedic three-dimensional reconstructions (600R) will optimize the environment for healing, reduce the surgical complication rate, increase the success rate as well as provide the possibility of in-hospital manufacture of the patient-specific orthopedic three-dimensional reconstructions (600R).

It is the novel and unique interaction of these simple elements which creates the process (123) and methods (123A, 123B, 123C) within the ambit of the present invention. Pursuant to Title 35 of the United States Code, descriptions of preferred embodiments follow. However, it is to be understood that the best mode descriptions do not limit the scope of the present invention.

### BRIEF DESCRIPTION OF THE DRAWINGS

FIG. 1 is a representation of sagittal (X-axis), coronal (Y-axis) and transverse planes (Z-axis) of a spinal segment.

FIG. 2 is a two dimensional sagittal X-plane portrayal of the spine, from the third lumbar (L3) vertebra (923) to the sacrum (930) including disks (940; L3-4), (942; L4-5), (944; L5-S).

FIG. 3 is a comparative perspective of first and second pedicles portraying traditional threaded pedicle screw (666) inserted into a target zone (40) of the first pedicle and a patient specific implant (600) implanted into a target zone (40) of the second pedicle. FIG. 3 is an axial Z-plane view of L3 vertebra (923) of FIG. 2 that references coronal slices C1 and C2.

FIG. 4 portrays a state-of-the-art pedicle screw.

FIGS. 4a-4c are two dimensional Y-axis portrayals of C1 and C2 slices of the L3 vertebra (923) of FIG. 3. FIGS. 4a-4c show prior art pedicle screw (666) traversing a first target zone (40) and threadless patient-specific pedicle fixation device (series 600, 620. 650, 690) traversing a second target zone (40). The C1 representation corresponds to the coronal slice near the entrance to the pedicle. The C2 representation corresponds to the area within the pedicle (902) with the smallest circumference.

FIG. 5 is a L5 axial plane view of a target zone (40) of L5 vertebra (925).

FIG. 6 is a L5 axial plane view of first and second target zones (40) of L5 vertebra (925).

FIG. 7 is an illustrative flow chart for the process (123) of making a patient-specific implant (600).

FIG. 8 is an illustrative flow chart for implantation and clinical evaluations of the patient-specific implant (600).

FIG. 9 illustrates a type of patient specific implant (600) that can be manufactured by the current process (123).

FIG. 9a illustrates a type of patient specific implant (600) that can be manufactured by the current process (123).

FIG. 10 illustrates a type of patient specific implant (600) that can be manufactured by the current process (123).

FIG. 10a illustrates a type of patient specific implant (600) that can be manufactured by the current process (123).

FIG. 11 illustrates a type of patient specific implant (600) that can be manufactured by the current process (123).

FIG. 12 illustrates a type of patient specific implant (600) that can be manufactured by the current process (123).

FIG. 11a illustrates a type of patient specific implant (600) that can be manufactured by the current process (123).

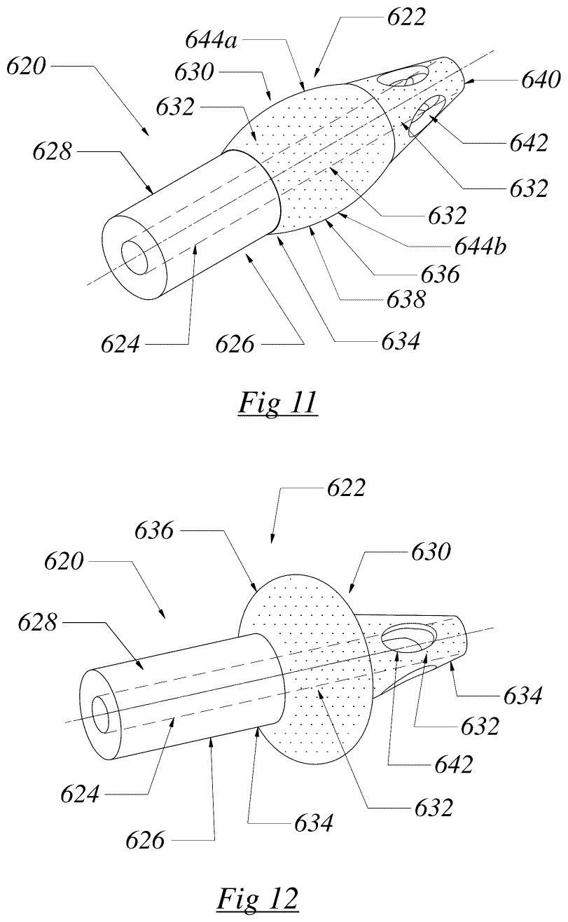

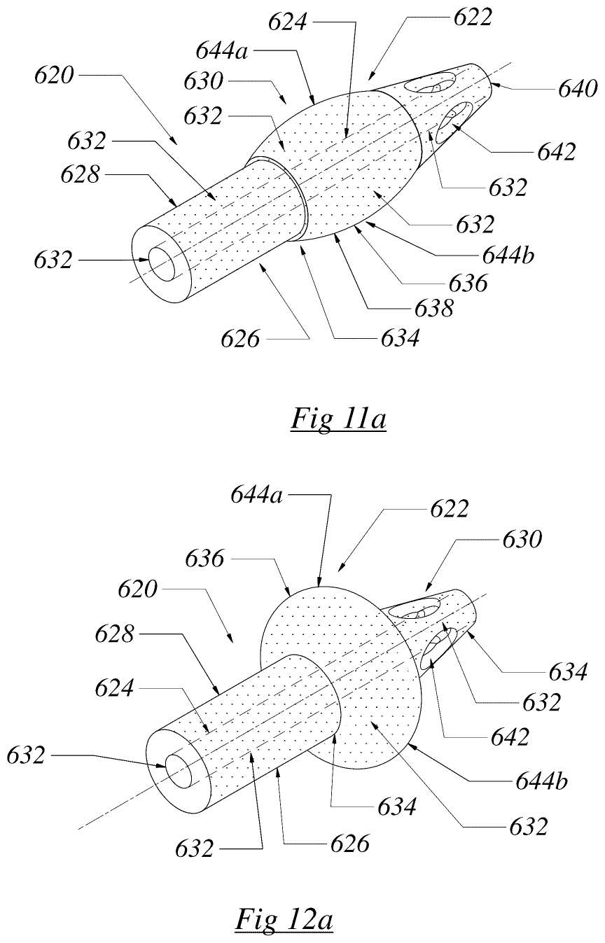

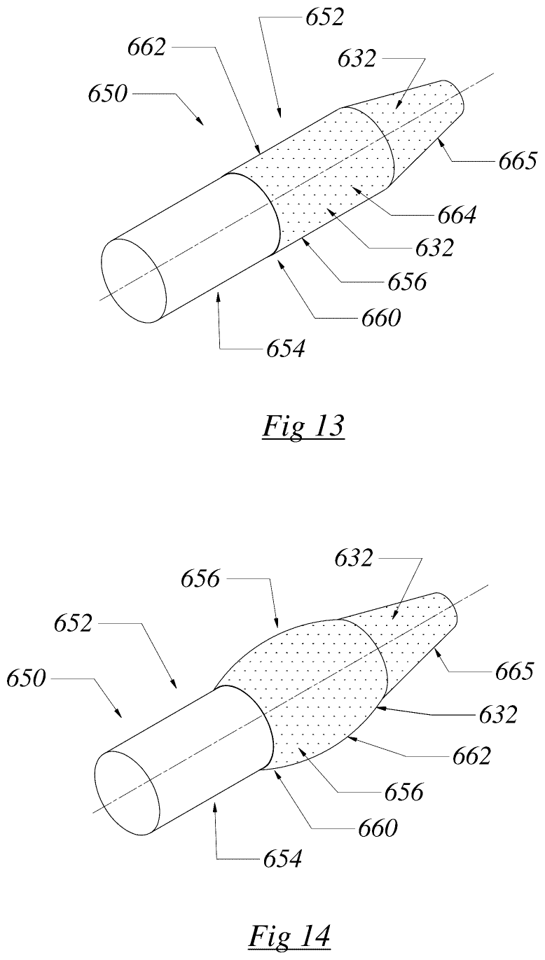

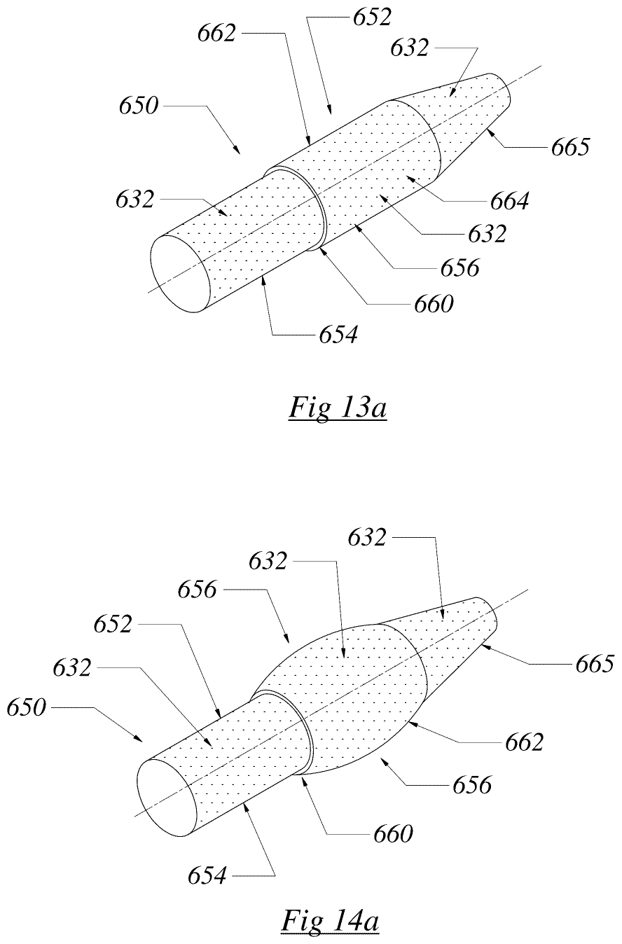

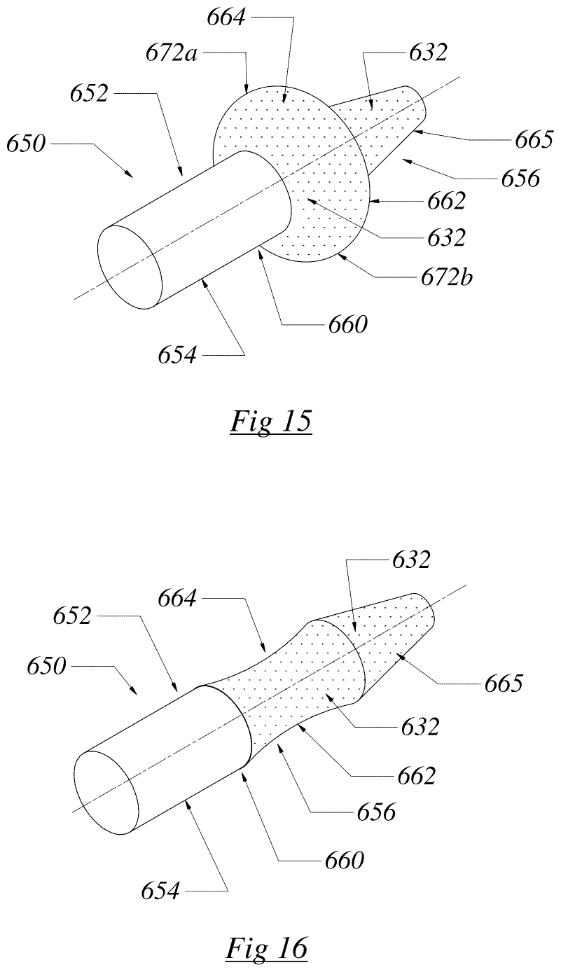

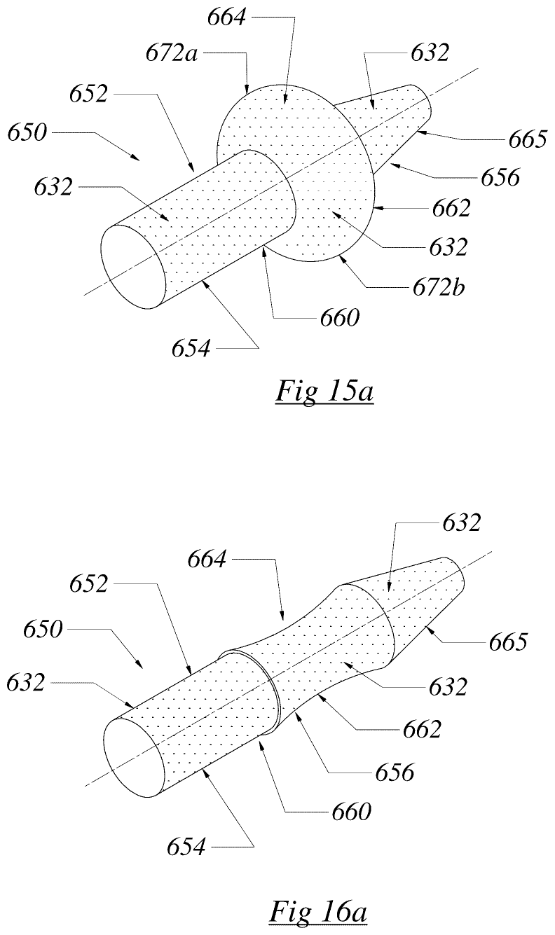

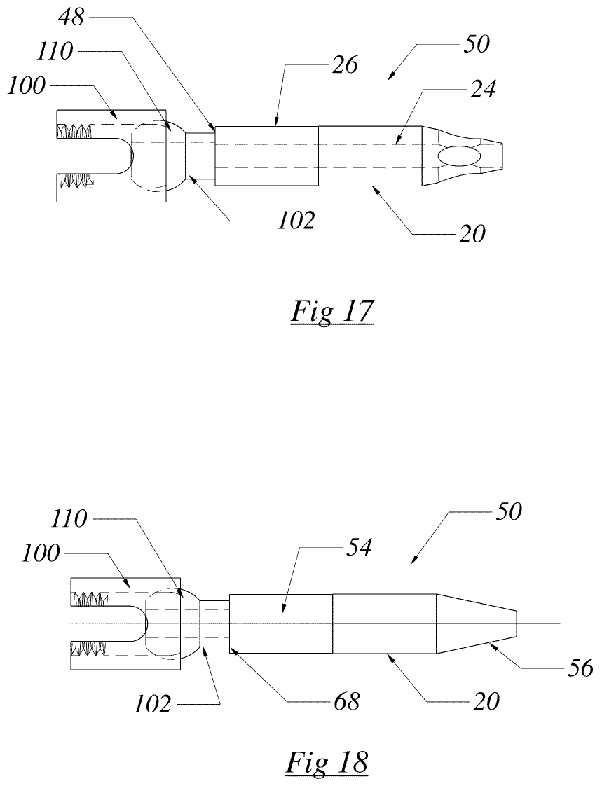

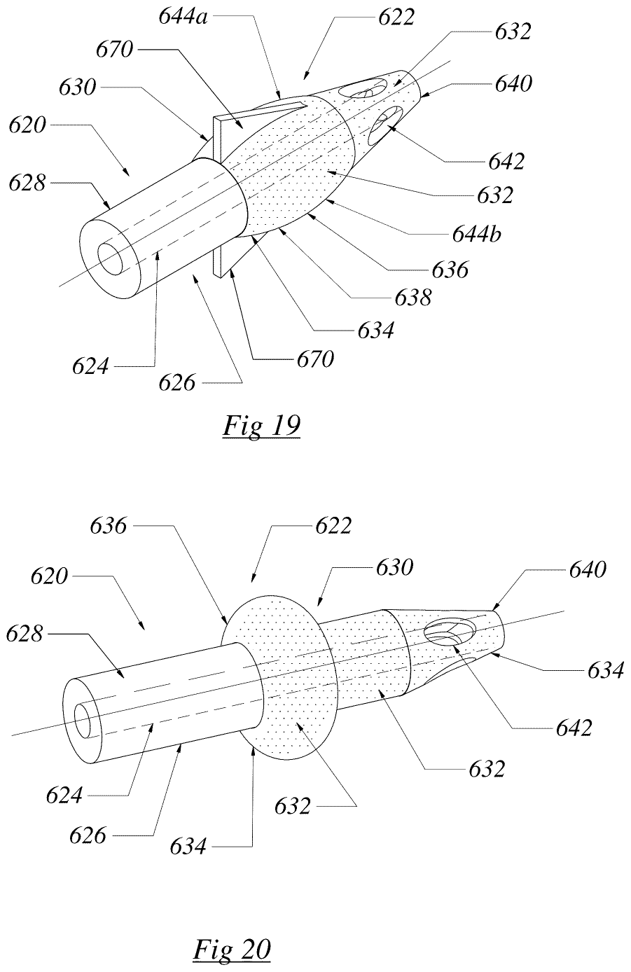

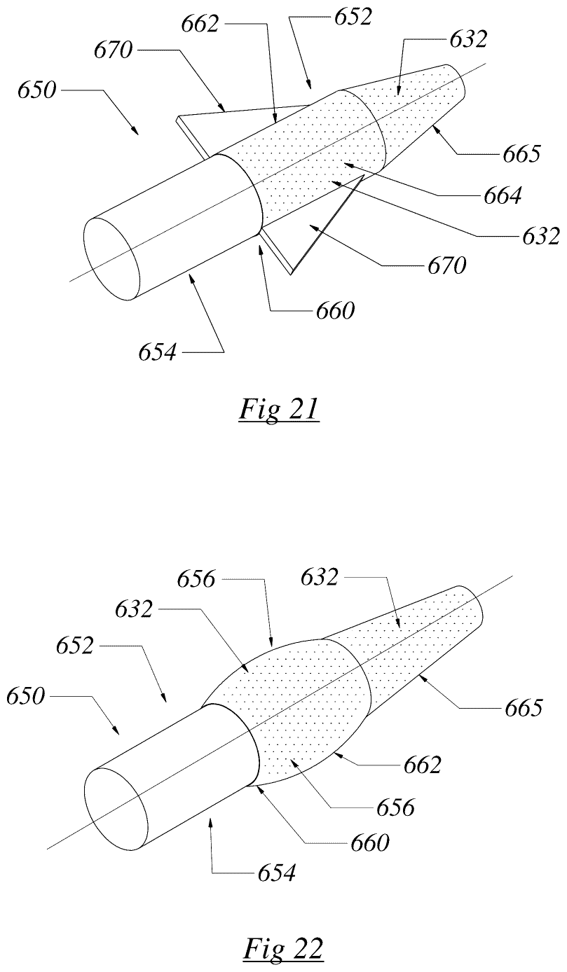

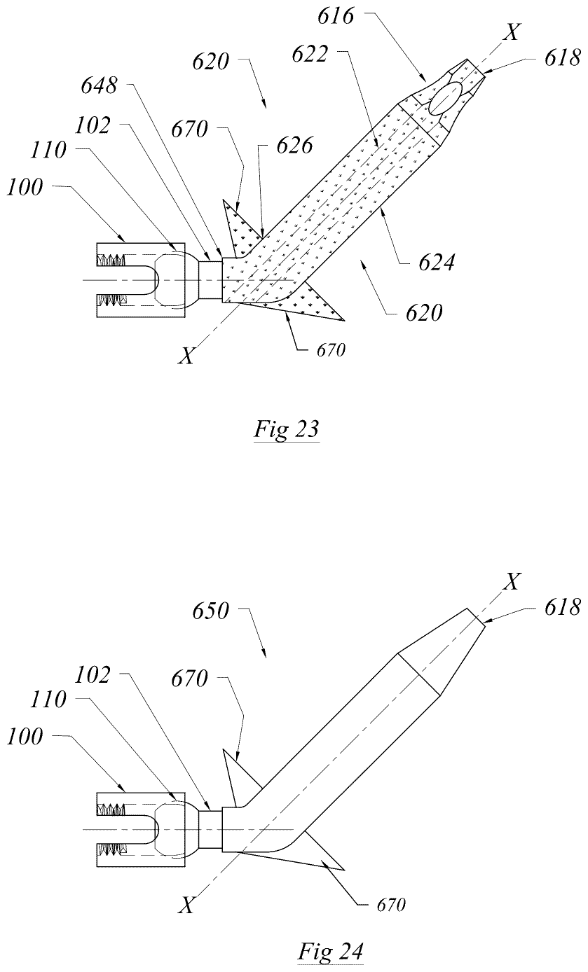

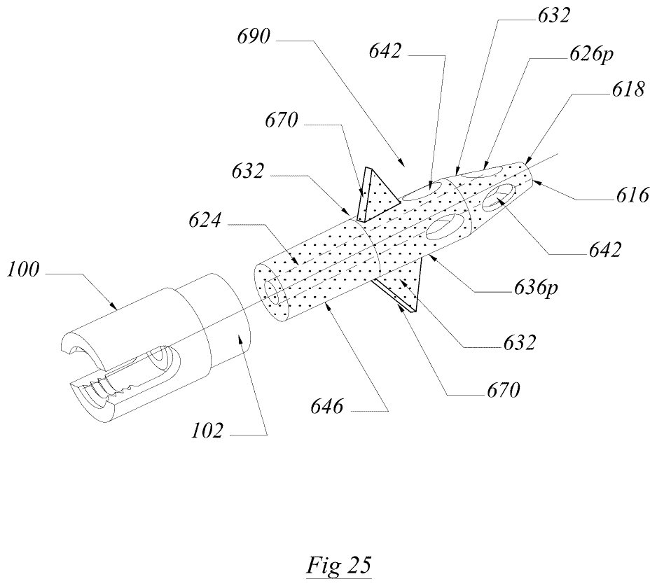

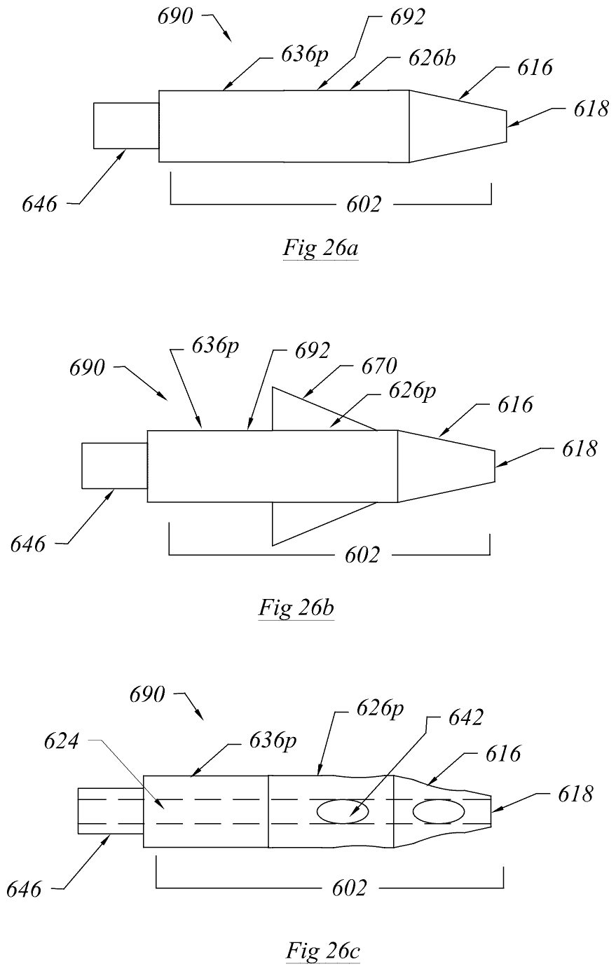

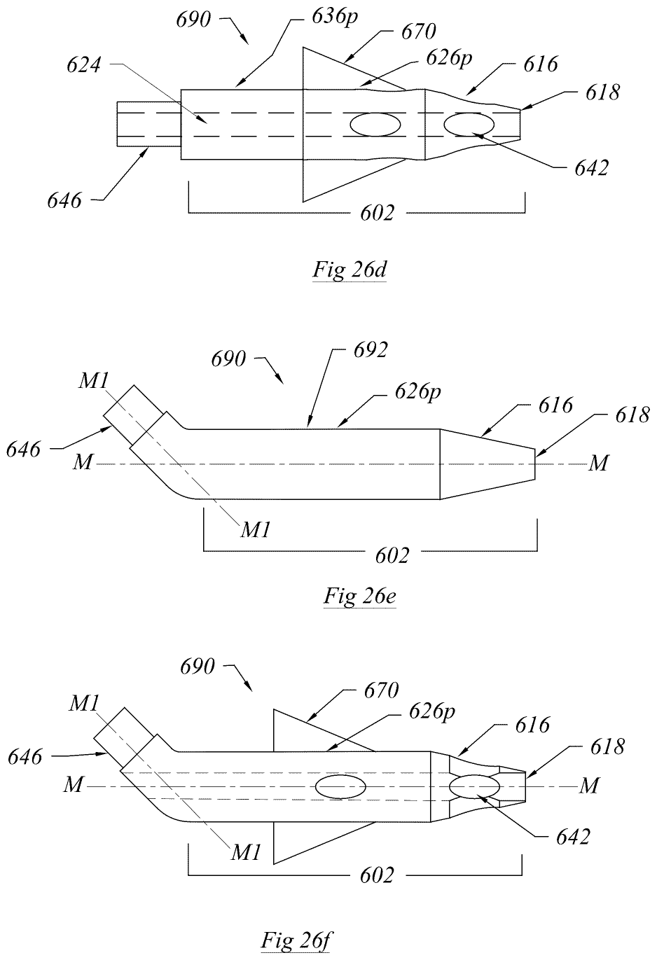

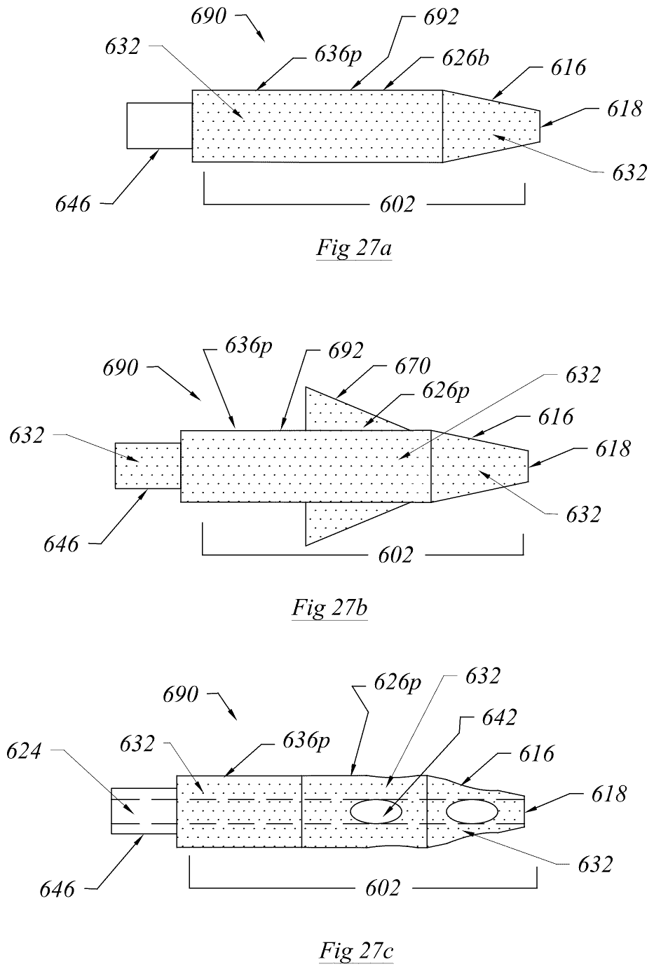

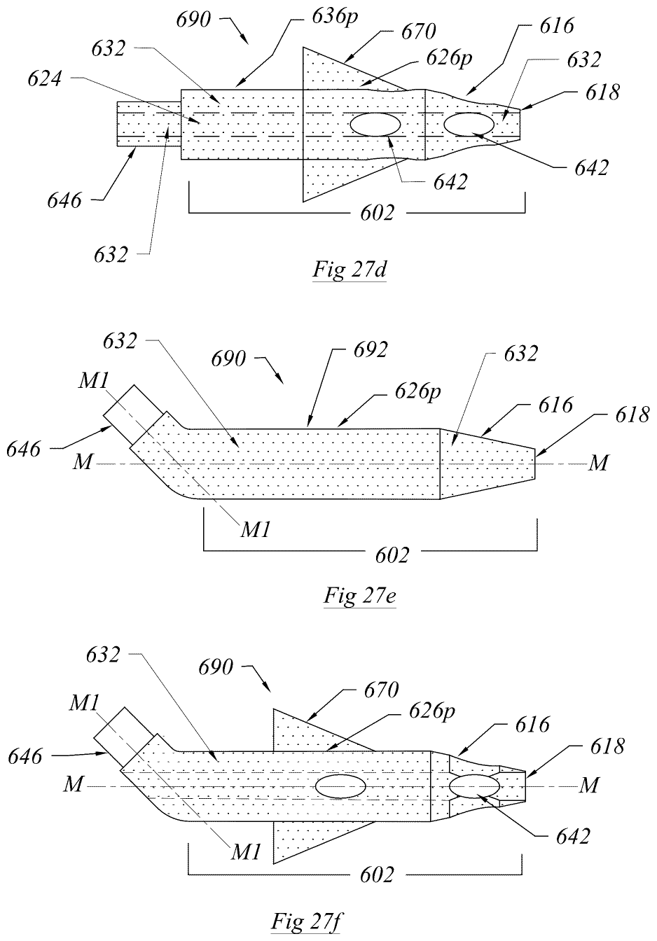

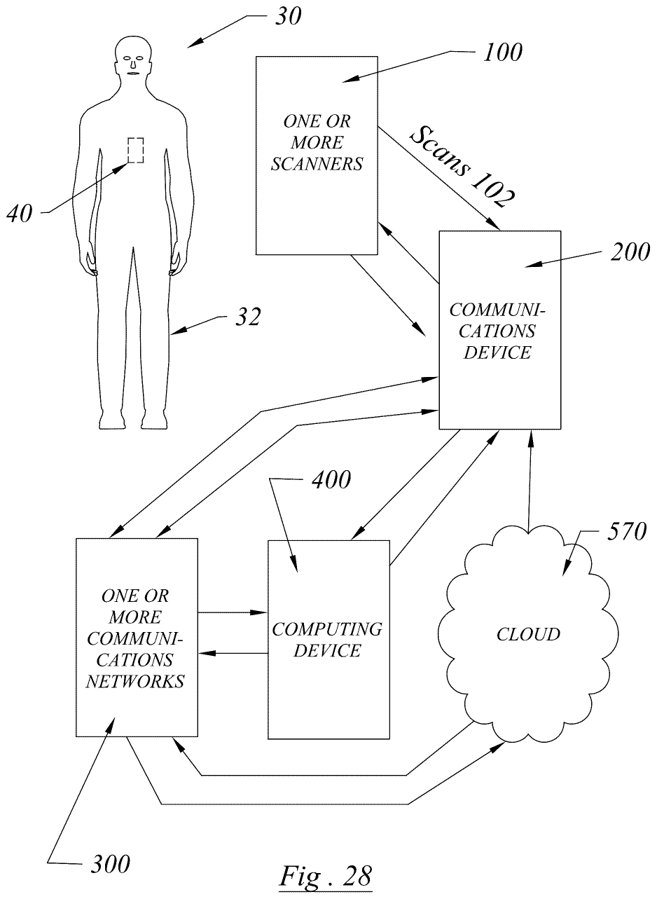

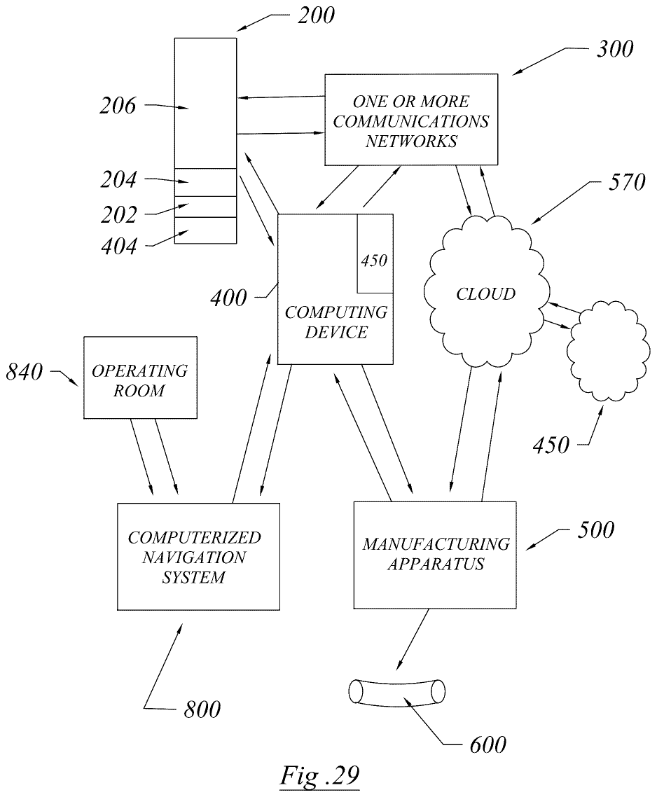

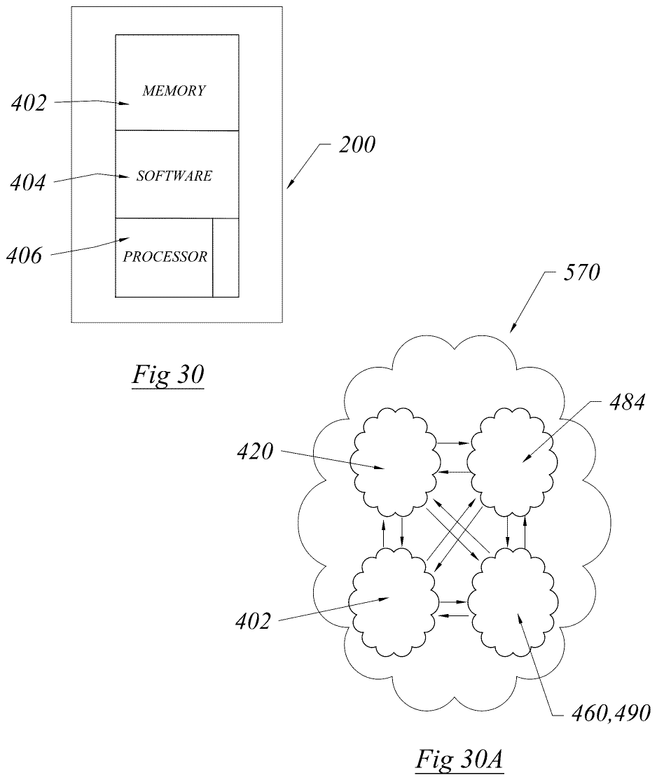

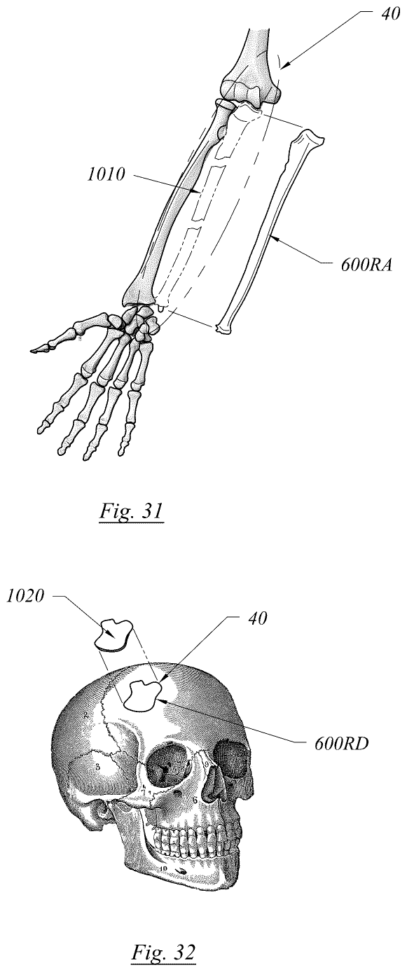

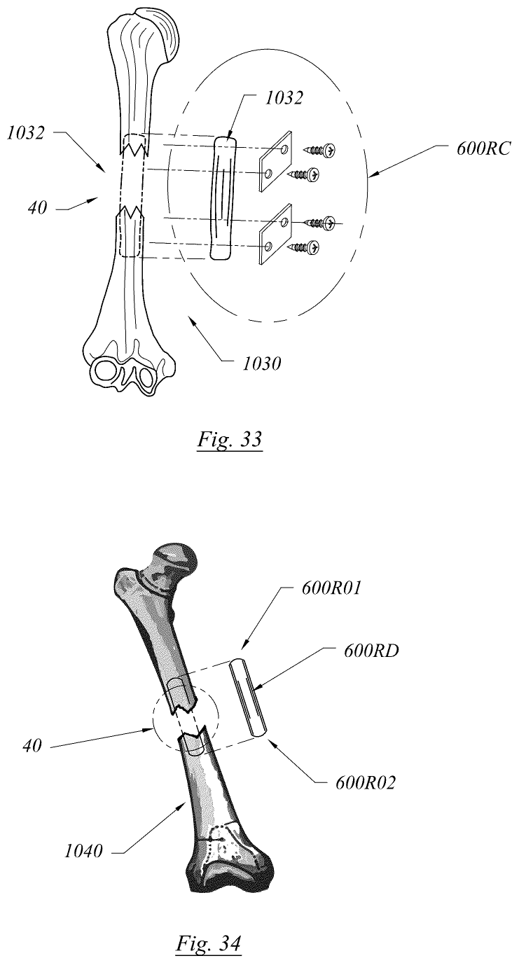

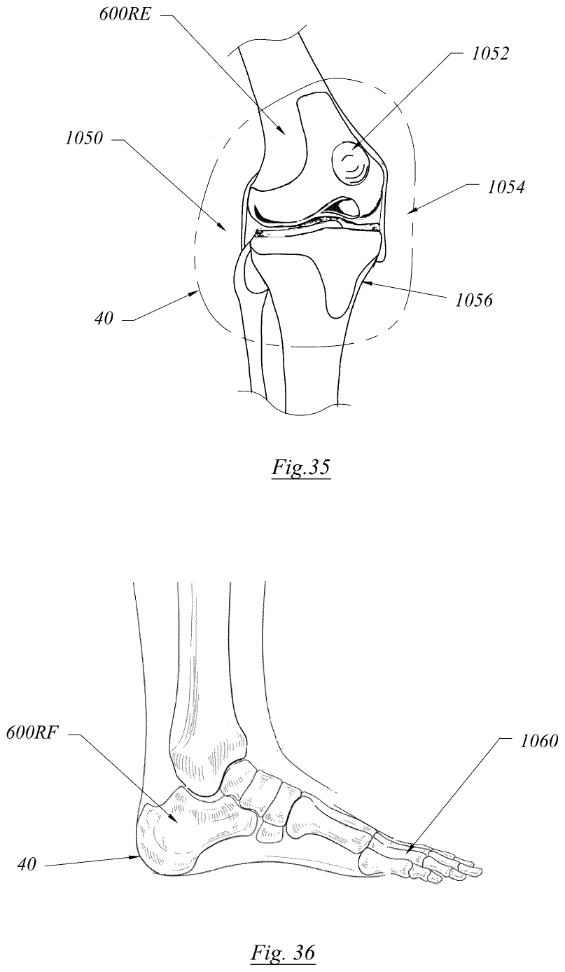

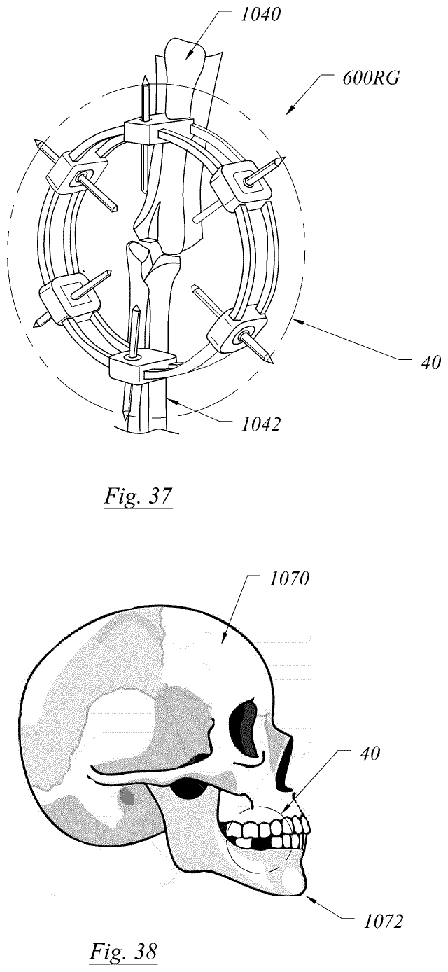

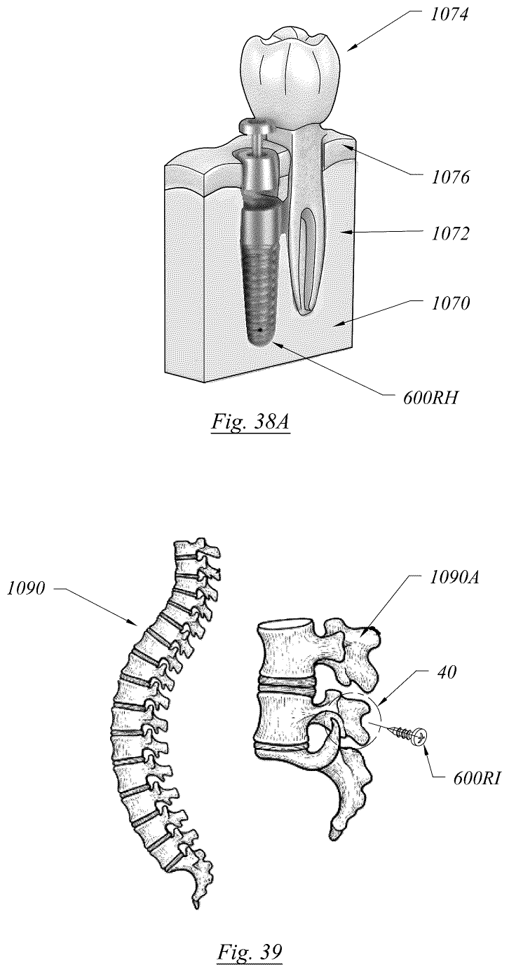

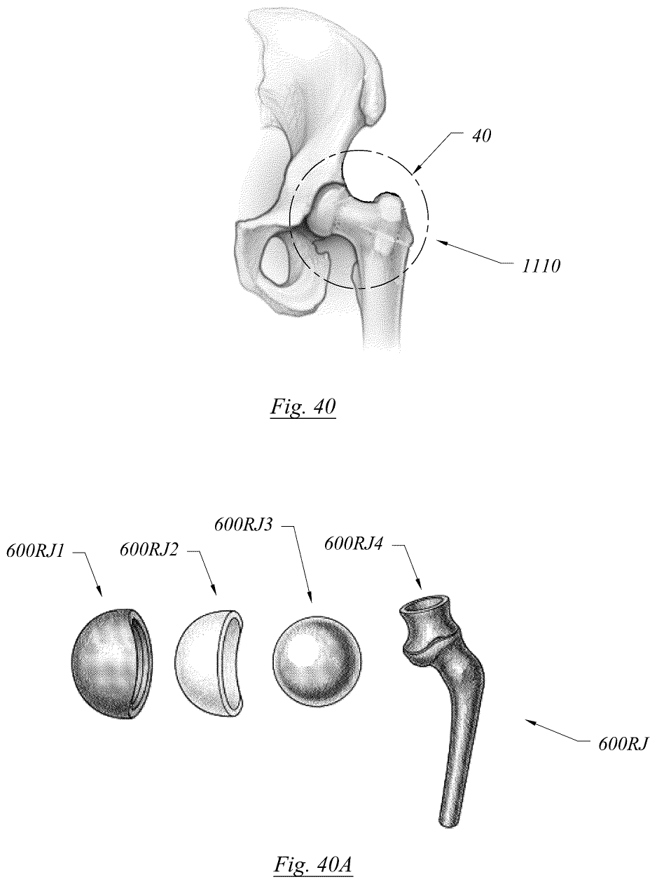

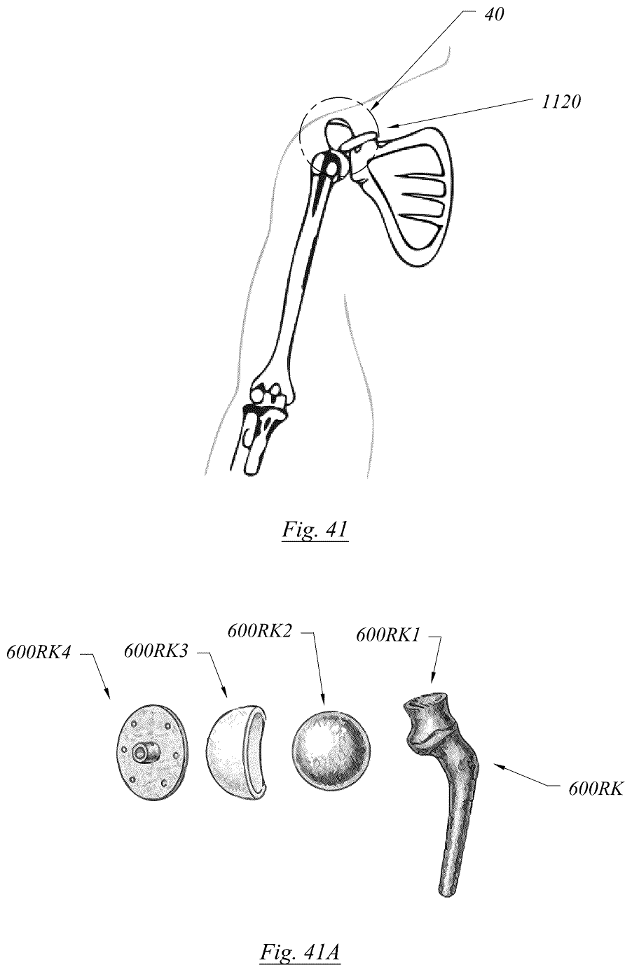

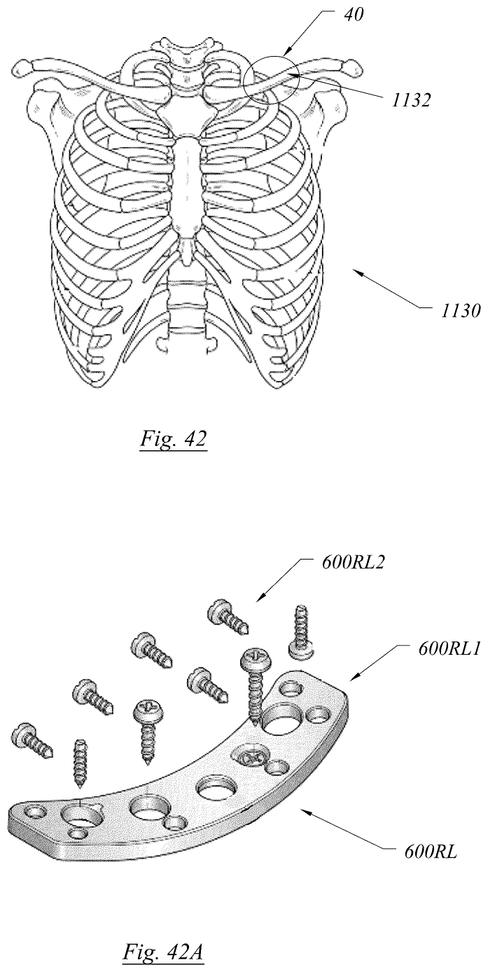

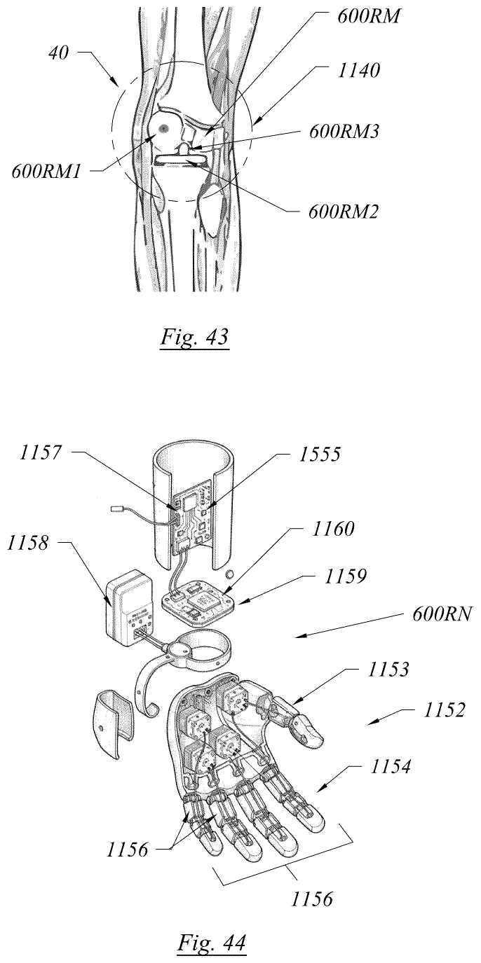

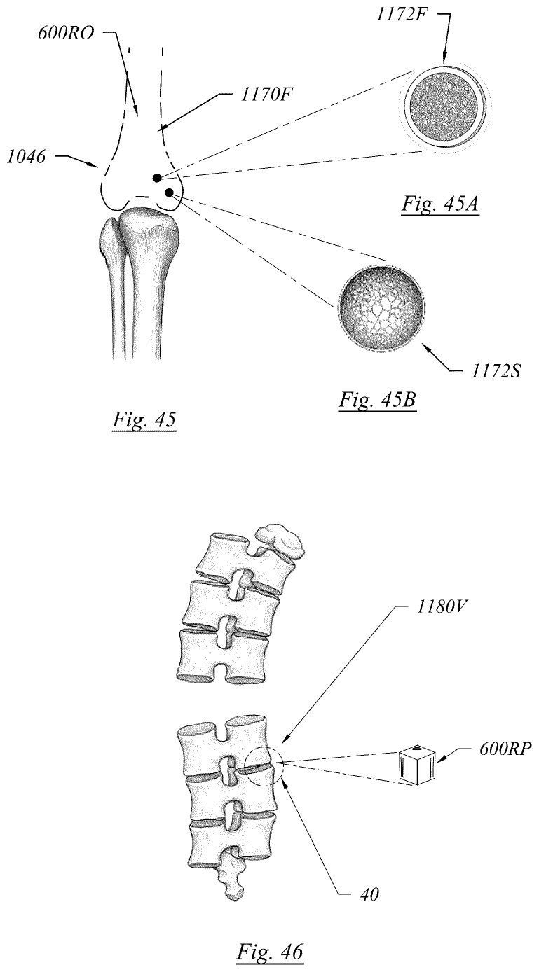

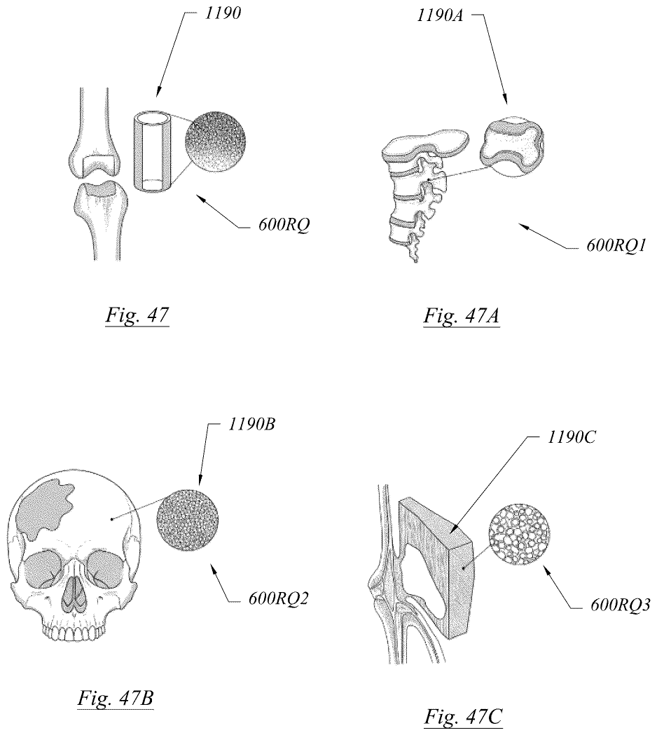

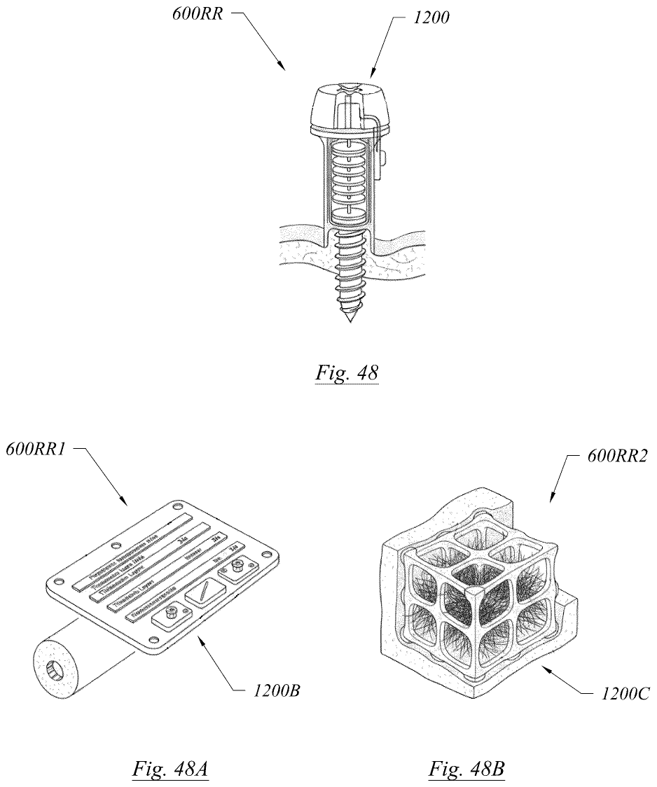

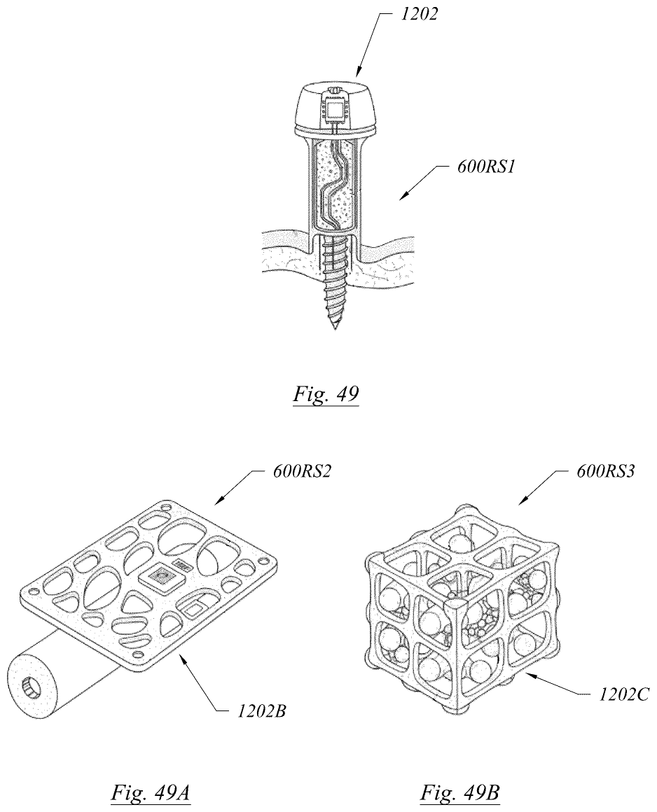

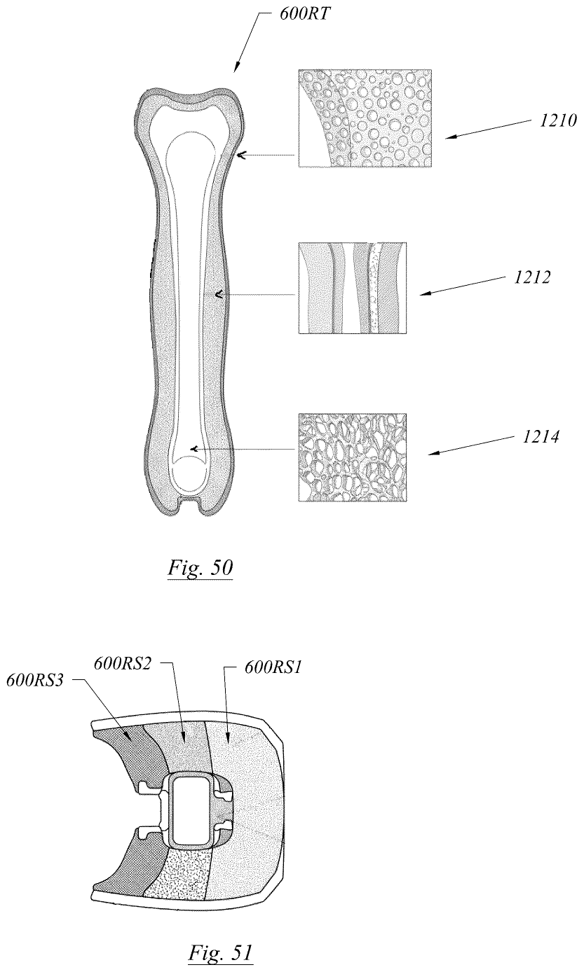

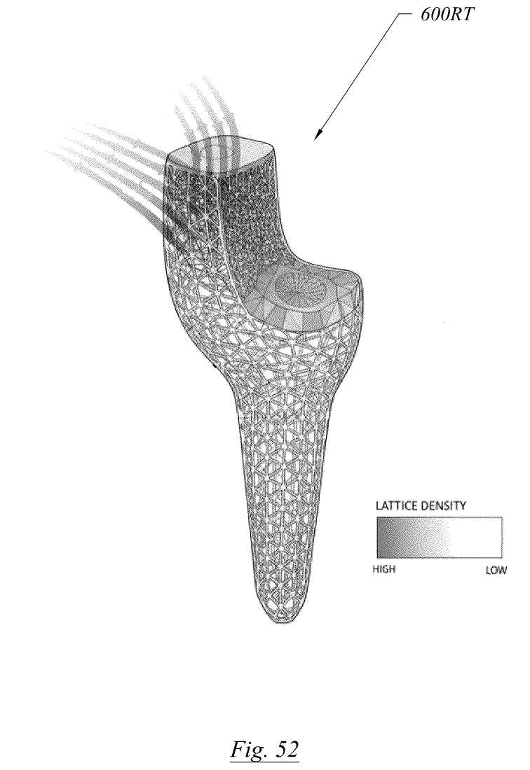

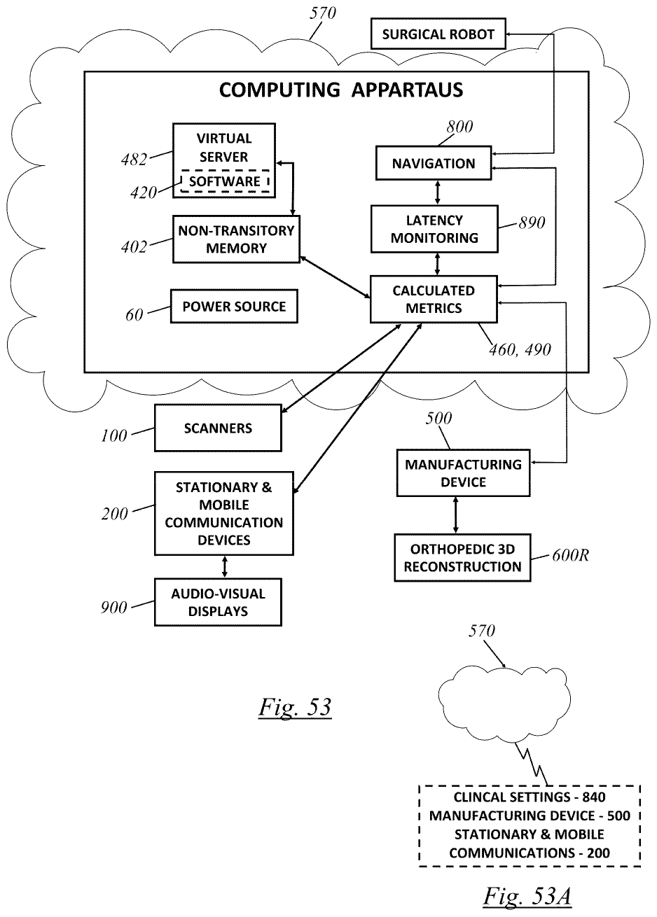

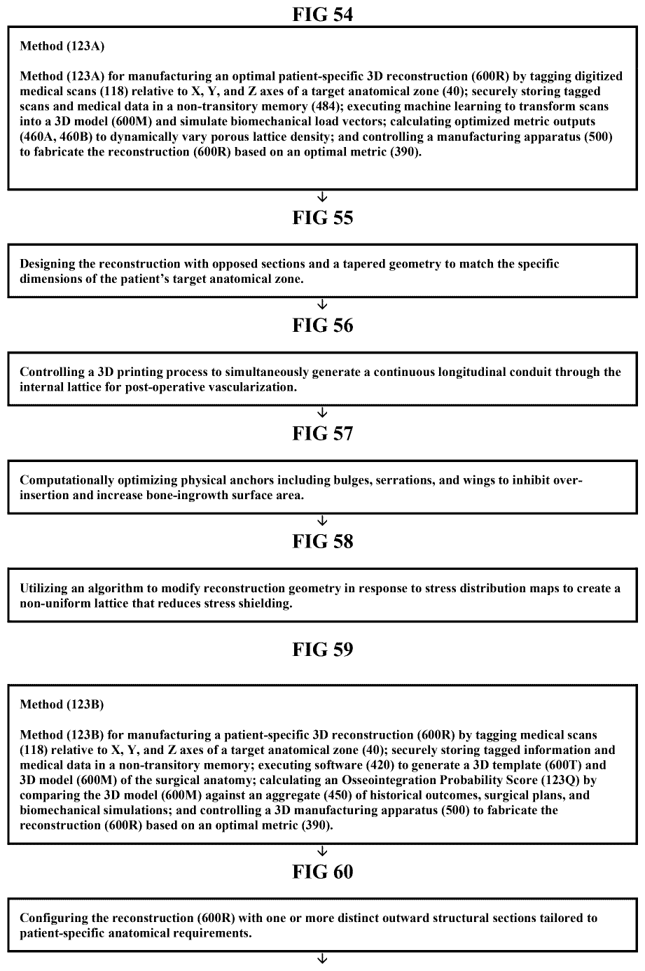

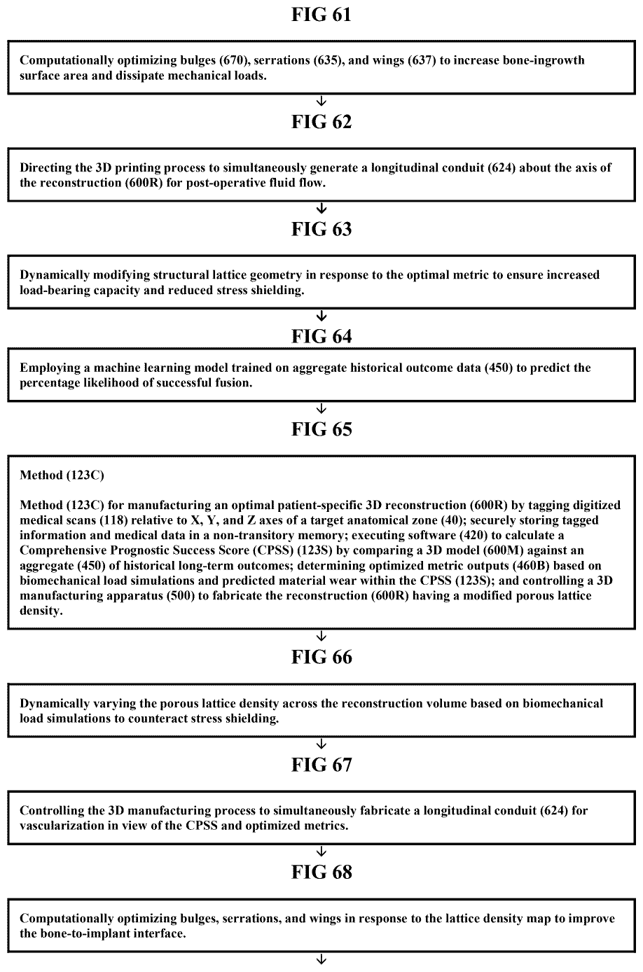

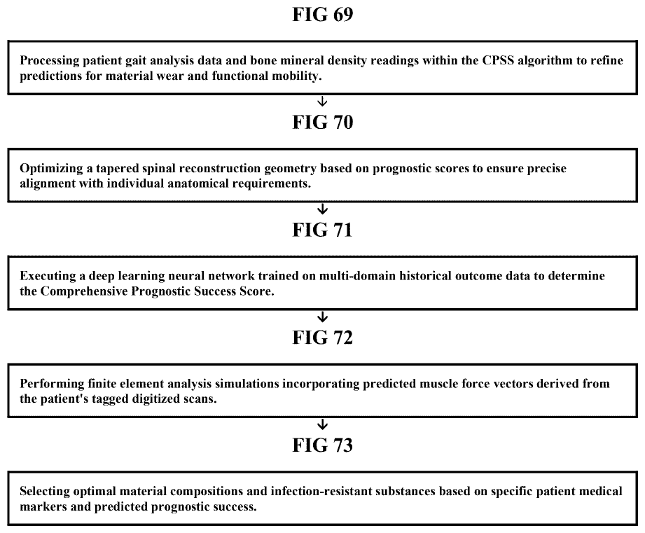

FIG. 12a illustrates a type of patient specific implant (600) that can be manufactured by the current process (123).

FIG. 13 illustrates a type of patient specific implant (600) that can be manufactured by the current process (123).

FIG. 14 illustrates a type of patient specific implant (600) that can be manufactured by the current process (123).

FIG. 13a illustrates a type of patient specific implant (600) that can be manufactured by the current process (123).

FIG. 14a illustrates a type of patient specific implant (600) that can be manufactured by the current process (123).

FIG. 15 illustrates a type of patient specific implant (600) that can be manufactured by the current process (123).

FIG. 16 illustrates a type of patient specific implant (600) that can be manufactured by the current process (123).

FIG. 15a illustrates a type of patient specific implant (600) that can be manufactured by the current process (123).

FIG. 16a illustrates a type of patient specific implant (600) that can be manufactured by the current process (123).

FIG. 17 illustrates a type of patient specific implant (600) that can be manufactured by the current process (123).

FIG. 18 illustrates a type of patient specific implant (600) that can be manufactured by the current process (123).

FIG. 19 illustrates a type of patient specific implant (600) that can be manufactured by the current process (123).

FIG. 20 illustrates a type of patient specific implant (600) that can be manufactured by the current process (123).

FIG. 21 illustrates a type of patient specific implant (600) that can be manufactured by the current process (123).

FIG. 22 illustrates a type of patient specific implant (600) that can be manufactured by the current process (123).

FIG. 23 illustrates a type of patient specific implant (600) that can be manufactured by the current process (123).

FIG. 24 illustrates a type of patient specific implant (600) that can be manufactured by the current process (123).

FIG. 25 illustrates a type of patient specific implant (600) that can be manufactured by the current process (123).

FIGS. 26a-26f illustrate patient specific implants that can be manufactured by process (123).

FIGS. 27a-27[j]f illustrate patient specific implants that can be manufactured by process (123).

FIGS. 28-30A disclose select embodiments of combinations of hardware and software associated with the steps of process (123) and methods (123A), (123B) and (123C) associated with human (32).

FIG. 31 illustrates shattered ulna (1010), target zone (40) an optimal orthopedic reconstruction (600RA) of a complete bone, manufactured in accordance with method (123A), (123B) or (123C).

FIG. 32 illustrates bone segment (1020) of a cranium, target zone (40) and an optimal orthopedic reconstruction (600RB) manufactured in accordance with method (123A), (123B) or (123C).

FIG. 33 illustrates a humerus (1030), target zone (40) and an optimal orthopedic reconstruction (600RC) manufactured in accordance with method (123A), (123B) or (123C).

FIG. 34 illustrates a femur (1040), target zone and an optimal orthopedic reconstruction (600RD) inside the medullary cavity of femur (1040) manufactured in accordance with method (123A), (123B) or (123C).

FIG. 35 illustrates a multi-component optimal orthopedic reconstruction (600RE) and target zone (40) for a knee (1050) including femur component (1052), tray (1054) and insert (1056) manufactured in accordance with method (123A), (123B) or (123C).

FIG. 36 illustrates a foot (1060) with an optimal orthopedic reconstruction (600RF) and target zone (40), e.g., calcaneus plate manufactured in accordance with method (123A), (123B) or (123C).

FIG. 37 illustrates a target zone (40) and an optimal orthopedic reconstruction (600RG), e.g., eternal fixator for the femur and tibia, manufactured in accordance with method (123A), (123B) or (123C).

FIG. 38 illustrates a mandible (1072) of skull (1070) with a missing tooth target zone (40).

FIG. 38A illustrates mandible (1072), healthy tooth (1074), gingiva (1076) and optimal orthopedic reconstruction (600RH) for implantation into target zone (40) manufactured in accordance with method (123A), (123B) or (123C).

FIG. 39 illustrates a vertebra (1090) including an exploded view of a thoracic section (1090A) with target zone (40) for receiving implantation of the optimal orthopedic reconstruction (600RI), e.g., pedicle screw (600RI), for implantation into target zone (40), manufactured in accordance with method (123A), (123B) or (123C).

FIG. 40 illustrates the acetabulofemoral joint (1110) and target zone (40).

FIG. 40A illustrates orthopedic reconstruction's (600RJ) components, e.g. acetabular cup (600RJ1), liner (600RJ2), (600RJ), femoral head (600RJ3) and femoral stem (600RJ4) creating reconstruction (600RJ) for implantation into target zone (40) manufactured in accordance with method (123A), (123B) or (123C).

FIG. 41 illustrates the humerus-scapula joint (1120) and target zone (40).

FIG. 41A illustrates potential types of able orthopedic reconstruction components humerus-scapula joint (1120) including humeral stem (600RK1), glenosphere (600RK2), humeral socket (600RK3) and metaglene (600RK4) creating reconstruction (600RK) for implantation into target zone (40) manufactured in accordance with method (123A), (123B) or (123C).

FIG. 42 illustrates a thoracic skeleton (1130), clavicle (1132) and target zone (40).

FIG. 42A illustrates orthopedic reconstruction (600RL) components, i.e., plate (600RL1) and screws (600RL2) for orthopedic reconstruction (600RL) manufactured in accordance with method (123A), (123B) or (123C).

FIG. 43 illustrates an elbow (1140), target zone (40) utilizing optimal orthopedic reconstruction (600RM) including bushings (600RM1, 600RM2) and pin (600RM3) manufactured in accordance with method (123A), (123B) or (123C).

FIG. 44 illustrates an optimal orthopedic reconstruction (600RN) of bionic hand (1152), movable thumb (1153) and fingers (1154), circuity (1555), tension wires (1156), sensors (1157), energy store (1158) and microcontroller (1159) including store firmware (1160) manufactured in accordance with method (123A), (123B) or (123C).

FIG. 45 illustrates an orthopedic reconstruction (600RO) for a portion of a femur (1140).

FIG. 45A portrays a cross-section of orthopedic reconstruction (600RO) including a first porous lattice (1172F) manufactured in accordance with method (123A), (123B) or (123C).

FIG. 45B shows a second porous lattice (1172S) for orthopedic reconstruction (600RO) manufactured in accordance with method (123A), (123B) or (123C).

FIG. 46 illustrates thoracic vertebra (1180V) with a defect at target zone (40) an orthopedic reconstruction (600RP) that is a regenerative scaffold manufactured in accordance with method (123A), (123B) or (123C).

FIGS. 47-47C illustrate orthopedic reconstructions (600RQ) that are distinct regenerative meshes (1190, 1190A, 1190B, 1190C) manufactured in accordance with method (123A), (123B) or (123C).

FIGS. 48-48 [C] B illustrate an orthopedic reconstruction (600RR) with nanogenerator components (1200, 1200B, 1200C) that can be included in orthopedic reconstruction (600RR) manufactured in accordance with method (123A), (123B) or (123C).

FIGS. 49-49 [C] B illustrate an orthopedic reconstruction (600RS) with biosensor components (1202, 1202B, 1202C) biosensor components that can included in orthopedic reconstruction (600RS) manufactured in accordance with method (123A), (123B) or (123C).

FIG. 50 illustrates orthopedic reconstruction (600RT) incorporating diverse densities for the benefit of the patient.

FIG. 51 illustrates orthopedic reconstruction (600RS) manufactured from a metal, metal-polymer combination and a polymer.

FIG. 52 illustrates orthopedic reconstruction (600RT) incorporating diverse densities for the benefit of the patient.

FIG. 53 is a flow diagram illustrating methodology of the Computerized Method of Making a Patient-Specific Orthopedic Reconstruction.

FIG. 53A illustrates tangible properties residing outside Cloud 570.

FIGS. 54-73 disclose computerized methods (123A, 123B, 123C) of making a patient-specific orthopedic reconstruction.

### DESCRIPTION OF PREFERRED COMPUTERIZED PROCESS FOR MAKING A PATIENT-SPECIFIC IMPLANT (123) AND COMPUTERIZED METHODS OF MAKING AN OPTIMAL PATIENT-SPECIFIC ORTHOPEDIC RECONSTRUCTION (123A), (123B) AND (123C)

Although the disclosure hereof is detailed to enable those skilled in the art to utilize the computerized process for making a patient-specific implant (123) and computerized methods (123A, 123B and 123C) of making optimal patient-specific orthopedic reconstructions (600R). The embodiments published herein merely exemplify select patient-specific implants and patient-specific orthopedic reconstructions. There are numerous available uses of the computerized optimal patient-specific orthopedic reconstructions (600R).

The computerized process for making a patient-specific implant (123) Whether identified as singular or plural, implant (600), screw (600), patient-specific spinal fixation device (600), pedicle fixation device (600), patient-specific fixation device (600) are spinal implants made by the current process (123) of making a patient-specific implant (600). As used herein, reference number (600) can reference a patient-specific implant (620, 650, 690) or any other patient-specific implant manufactured by the current process (123) of making a patient-specific implant (600). Any reference to process (123) signifies the computerized process for making a patient-specific implant (123).

With regard to vertebra (920): each vertebra (920) has body (901), two pedicles (902a, 902b), two transverse processes (903a, 903b), two mammillary processes (904a, 904b), two lamina (905a, 905b), and one spinous process (906). Depending on the context of the sentence, reference numbers (902, 902a, 902b) refer to one or more pedicles; reference numbers (904, 904a, 904b) refer to one or more mammillary processes; and reference numbers (905, 905a, 905b) refer to one or more lamina. In the most general sense, the present invention is a process (123) of making a patient-specific pedicle fixation device (600) for a target zone (40) within the patient's (30) vertebra (920). Preferred embodiments of the pedicle fixation device (600) can have four distinct regions. First region (616) guides the pedicle fixation device (600) into its desired location. First Region (616) has a blunt leading edge in front and connects posteriorly to second region (626p). The respective heights and respective widths of the patient-specific fixation device (600) in first region (616) are less than any height or width in second region (626p). The patient-specific pedicle fixation device (600) in second region (626p) can have dimensions that maximize surface contact area (602) with the geometric dimensions (42) of target zone (40) and the subcortical bone within the pedicle (902). Second region (626p) is connected to first region (616) and third region (636p). Third region (636p) corresponds to the area in between pedicle (902) and the mammillary process (904). Wings, bulges and other process (123) metric output (408) customized features (637) can be added to third region (636p). Process (123) metric output (408) can make bends or curves in third region (636p); the bends or curves can be relative to the shared midlines (M1-M1) of the third region (636p) and fourth region (646) and the shared midlines (M-M) of first region (616) and second region (626p). In select preferred embodiments of the current patient-specific implant (600), shared midlines (M1-M1) of the third region (636p) and fourth region (646) are offset of up to about 45 degrees from the shared midlines (M-M) of first region (616) and second region (626p) to create an angled patient-specific implant (600). The angled patient-specific implant (600) can improve ease of interconnection with spinal devices distinct from patient-specific implant (600). Fourth region (646) connects with third region (636p). Fourth region is connectable to devices distinct from patient-specific fixation device (600). Fourth region (646) can be connected with a post (639) to accommodate side loading connectors or a tulip head (not shown) to serve as a top loading connector. Among the plethora of patient-specific pedicle fixation devices (600) that could be made by the current process (123), select examples follow. In view of the patient's target zone (40), process (123) can manufacture additions or subtractions to patient-specific device (600).

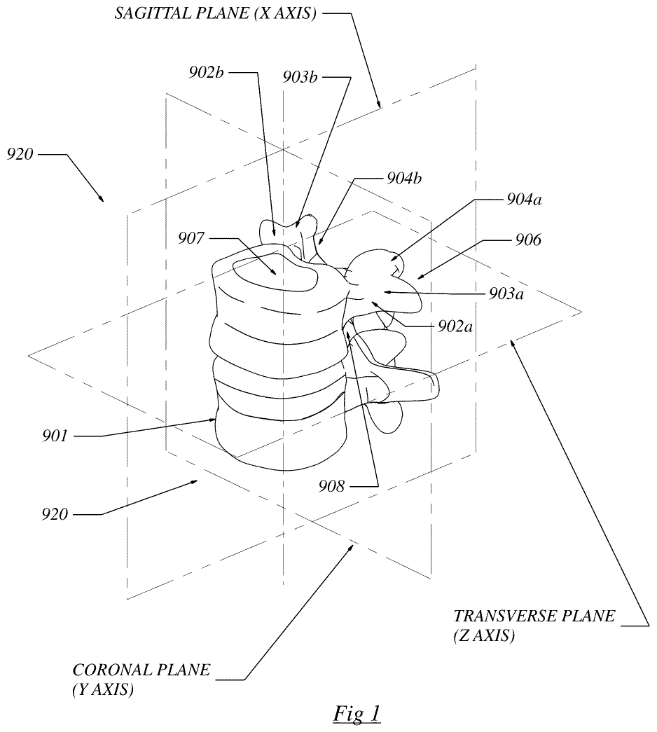

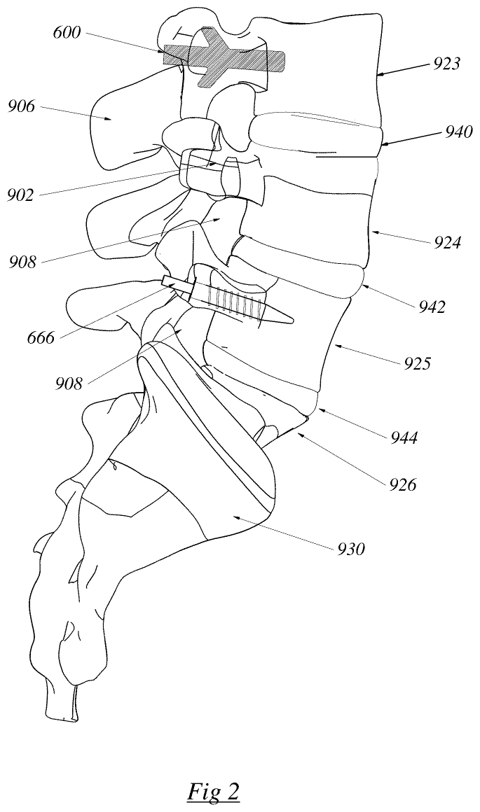

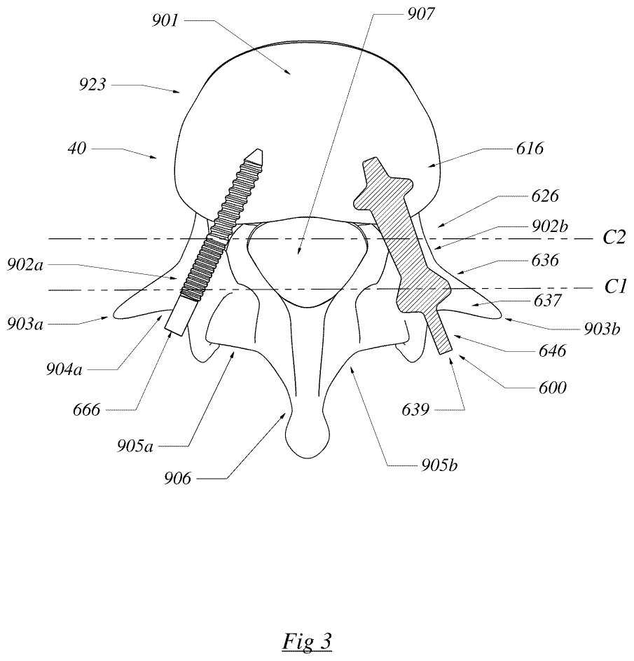

FIG. 1 is a representation of sagittal (X-axis), coronal (Y-axis) and transverse planes (Z-axis) of a spinal segment. Among other things, vertebra (920) has a body (901), two pedicles (902a, 902b), two transverse processes (903a, 903b), two mammillary processes (904a, 904b), two lamina (905a, 905b), and one spinous process (906). The central spinal canal (907) contains the spinal cord and cauda equina. The neuroforamin (908a. 908b) allow nerves and blood vessels to enter and exit the spinal canal (907). FIGS. 2 and 3 are two dimensional portrayals of a spine.

FIG. 2 is a sagittal X-plane portrayal from the third lumbar (L3) vertebra (923) to the sacrum (930) including disks (940; L3-4), (942; L4-5), (944; L5-S1). A patient-specific pedicle fixation device (600) is positioned inside the target zone (40) of L3 (923).

FIG. 3 is an axial Z-plane view of L3 vertebra (923) of FIG. 2 providing reference to coronal slices C1 and C2. A traditional threaded pedicle screw (666) traverses a first target zone (40) and a patient-specific implant traverses a second target zone (40).

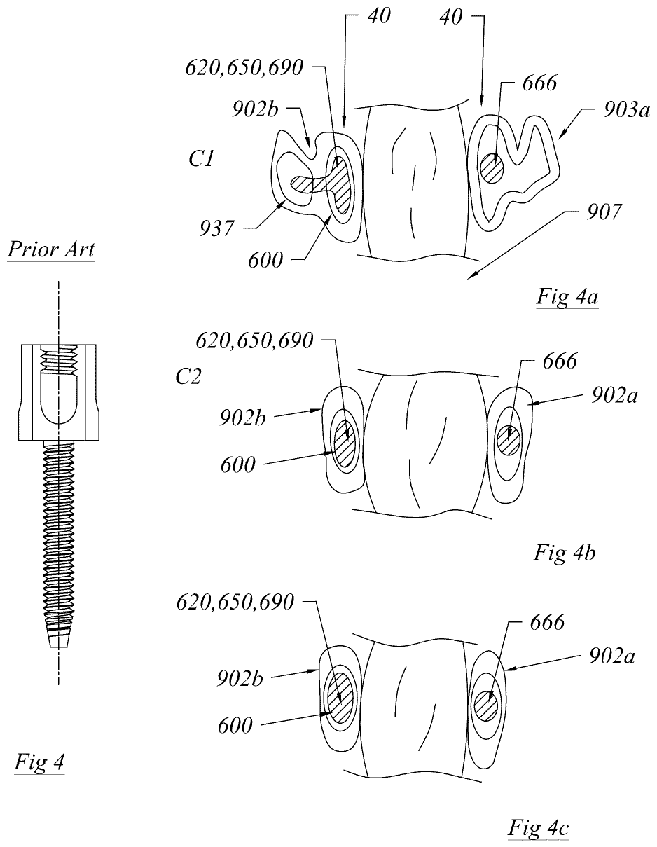

FIG. 4 portrays a state-of-the-art pedicle screw. FIGS. 4a-4b are coronal Y-plane views of the L3 vertebra (923) shown in FIG. 3. A prior art pedicle screw (666) is traversing (C1) on the left sides of FIGS. 4a-4b. A threadless patient-specific pedicle fixation device (600) is traversing (C1) on the right sides of FIGS. 4a-4b. The oval shape of patient-specific pedicle fixation devices (600) will have greater surface contact areas with subcortical bone inside the pedicle (902) than previous art pedicle screws (666).

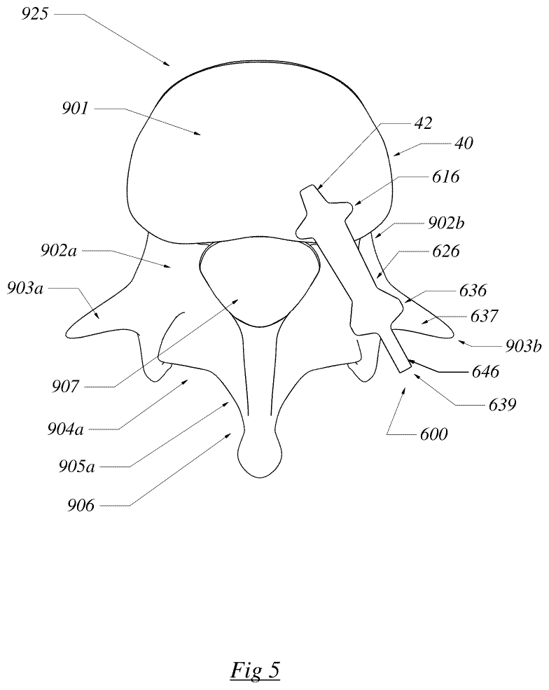

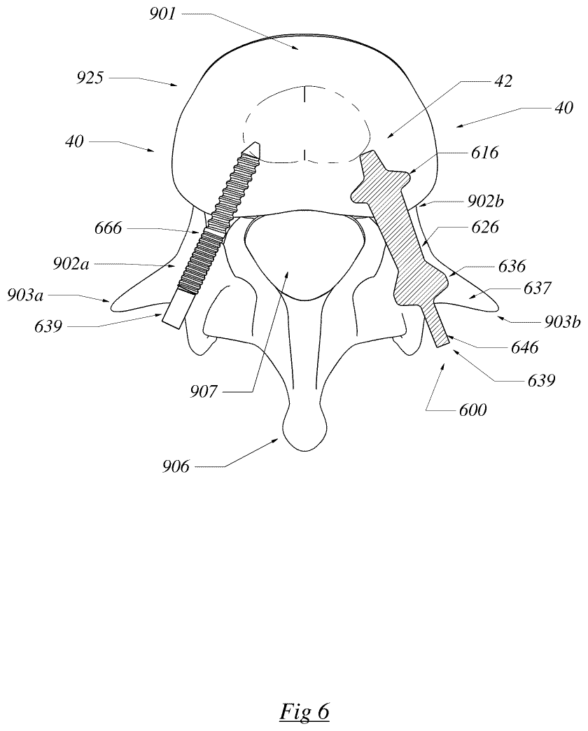

FIG. 5 is an axial plane view of a target zone (40) of L5 vertebra (925). L5 pedicles (902) have larger diameters than the L3 pedicles. The medial-lateral insertion angles are also larger, averaging 45 degrees towards the midline. The current process' metric output (408) can make threadless patient-specific fixation device (600) with two enlargements or wings (637) in third region (636p) and surface treatments (632) in first, second and third regions (616, 626p, 636p). It is believed that surface treatments (632) facilitate bone ingrowth. Wings (637), rings or circumferential enlargements in the third region (636p) can limit the depth of insertion, optimize the alignment of the subcortical bone within pedicle (902) in second region (626p) of the patient-specific fixation device (600), increase the surface area for subcortical bone ingrowth from the transverse process (903) and lamina (905), increase the surface area for bone ingrowth of the inter-transverse process fusion, and provide a larger surface area to disperse loads transmitted from spinal levels adjacent to the patient-specific fixation device (600).

FIG. 6 is an axial plane view of first and second target zones (40) of L5 vertebra (925). A prior art threaded pedicle screw (666) traverses the target zone's (40) pedicle (902a). A threadless patient-specific pedicle fixation device (600) traverses the target zone's (40) pedicle (902b). In addition to wings (637), metric (408) of the process (123) for making a patient-specific spinal fixation device (600) manufactured a bend in third region (636p). As shown, first region (616) and second region (626p) are centered about shared midline (M-M) while midline (M1-M1) of third region (636p) and fourth region (646) includes an offset angle of about 30 degrees relative to shared midline (M-M) of first region (616) and second region (626p). This medial-lateral angulation adjustment can reduce the dissection area required for implantation and the risk for a post-operative infection. This personalized angulation adjustment can also simplify connection with other spinal devices.

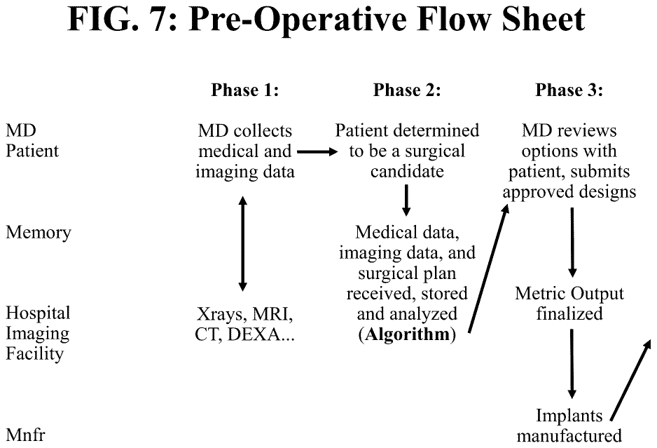

FIG. 7 is an illustrative flow chart for the process (123) of making a patient-specific implant (600) during the pre-operative period.

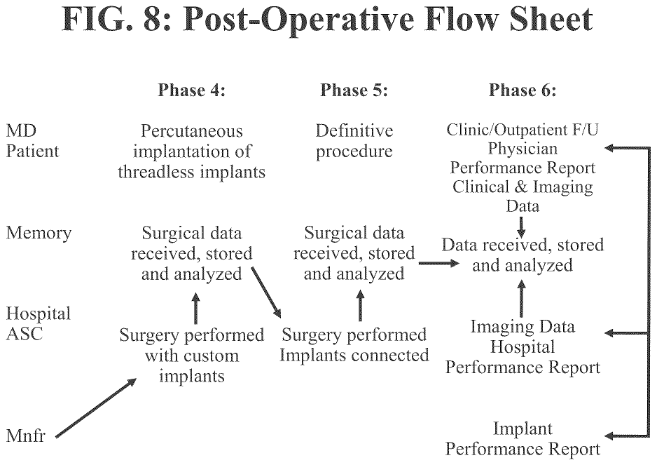

FIG. 8 is an illustrative flow chart for the implantation and clinical evaluations of the patient-specific implant (600).

Within the scope of the current process (123), it has been advantageously discovered that the series 600 (620, 650, 690) patient-specific implants (600) can have lengths from about 20 to about 75 millimeters; polyaxial heads (100) can have lengths of from about 10 millimeters to about 25 millimeters. However, other patient-specific implants within the scope of the current process (123) can have different measurements. Preferred embodiments of process (123) can utilize additive techniques, such as 3D printing, to build the device out of microscopic metal particles. Implants can be built particle by particle over the height, length and width of the patient-specific implant. Particles can be fused together to maximize density and create smooth (external) surfaces exposed to a surgical environment. It is believed that densely fused particles improve a 3D printed implant's biomechanical strength. Densely fused particles can also provide the 3D printed implant a smooth surface over which connectors and other devices can be attached. In contrast, particles fused together in clumps can create a rough or a porous texture. Rough or porous surfaces may sacrifice implant strength for the facilitation of bone ingrowth. In select preferred embodiments, rough surfaces can be included with a conduit of the current implant. Bone ingrowth into the patient-specific implant can increase the implant's biomechanical strength and allow living bone or other tissues to grow into available spaces.

Preferred embodiments of process (123) can utilize subtractive manufacturing methods that start with a solid block of metal or other composition acceptable in the art that is larger in height, length and width than the patient-specific implant. Subtractive manufacturing removes portions of the block to create the preselected dimensions of the implant. Abrasive particles, lasers and/or chemical treatments can be used to roughen the surface of the implant. During subtractive manufacturing of the implant, its total size decreases with each intervention.

By way of illustration, patient-specific implants can be created by the combination of subtractive and additive manufacturing techniques where an additive rough surface is added to the implant initially created by subtractive manufacturing. For example, press-fit total hip and press-fit total knee implants can be manufactured with the combination of subtractive and additive manufacturing techniques.

It is believed that rough surfaces can assist with long term fixation of the patient-specific implants by allowing more bone ingrowth onto and/or into the implant. Within the scope of the current process (123), either additive or subtractive means or a combination thereof can create the rough surfaces for any exposed surface of the patient-specific implants. For the purposes of this Application, “rough surfaces” are defined as, “biocompatible surfaces created by additive and/or subtractive means on any surface of the patient-specific implant that can facilitate ingrowth or interdigitation of the host tissues with the implant.”

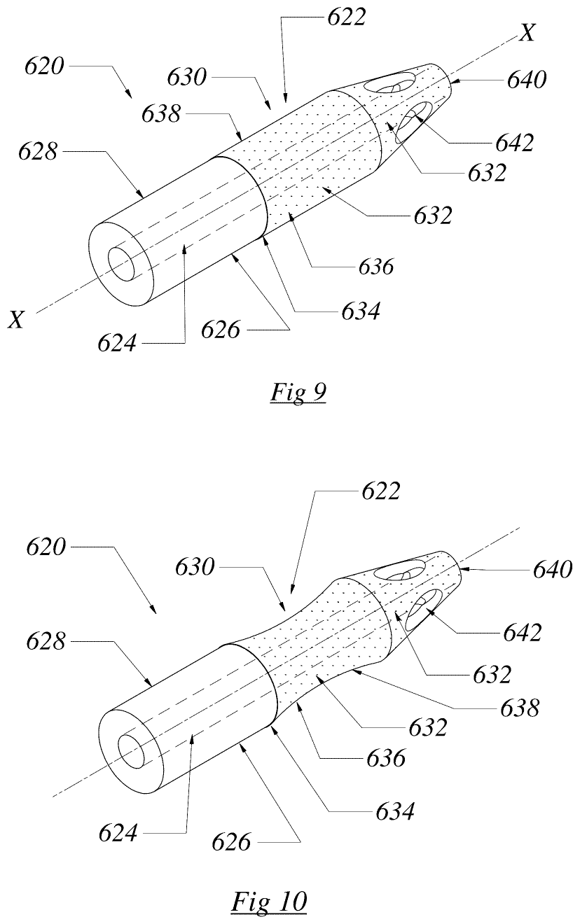

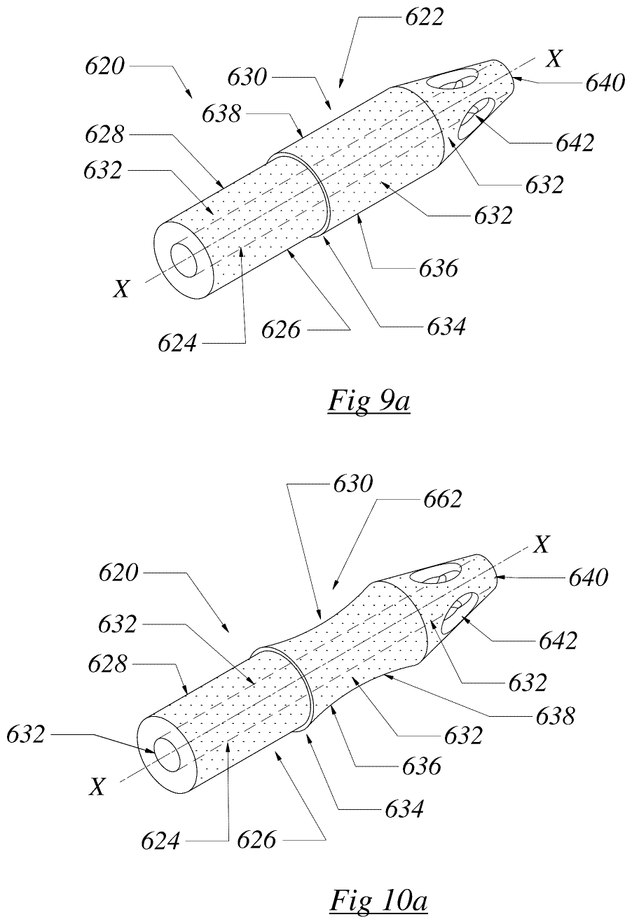

Returning to the series 600 implants; FIGS. 9-27[j] f illustrate types of patent specific implants (620, 650, 690) that can be manufactured by process (123). In a preferred embodiment, patient-specific implant (620) is provided with cannula (622). Cannula (622) includes conduit (624) adapted to carry one or more biocompatible substances. Conduit (624) traverses an entire length of a longitudinal axis of cannula (622).

Barrier (626) surrounds conduit (624). Barrier (626) can be provided with a first cylindrical section (628) with layers of rough surfaces (632) and a second section (630), adjacent to the first cylindrical section (628), with more layers of rough surfaces (632) than the first cylindrical section (628). First cylindrical section (628) includes a first diameter (634) merged with the second section (630). Second section (630) includes first segment (636) proximate the first cylindrical section (628). A portion of a cross-sectional diameter (638) of the first segment (636) is less than, equal to or greater than the first diameter (634). Second segment (640) is merged and connected with the first segment (636). Second segment (640) is interrupted by one or more openings (642) allowing interactions between conduit (624) and a surgically created environment proximate patient-specific implant (620).

Among other things, rough surfaces (632) can include micropores, metal, abrasive particles, dense particles or clumps of particles. When for the surgically created cavity dictates, first segment (636) is cylindrical and second segment (640) is conical. When the surgically created cavity dictates, first segment (636) is biconvex and the second segment (640) is conical. First segment (636) can be provided with a greater length than curved lengths of each opposed convex sides (644a, 644b). First segment (636) can also be ovoid. When the surgically created cavity dictates, first segment (636) is biconcave and second segment (640) is conical.

As shown in FIG. 19, when the surgically created cavity dictates, patient-specific implant (620) can be provided with wings or bulges (670).

Select preferred embodiments of patient-specific implant (620) shown in FIG. 17 can be provided with polyaxial head (100) connected to spheroid connector (102) that is attached to first side (648) of barrier (626) opposite the second segment (640). As shown, conduit (624) reciprocates with channel (104) of spheroid connector (102). Polyaxial head (100) includes receptacle (110) that snap fits/locks over spheroid connector (102).

As shown in FIG. 24, spheroid connector (102) can be offset of up to about 45 degrees from the longitudinal axis (X-X) of patient-specific implant (620).

FIGS. 13-16a are lateral perspectives of patient-specific implant (650). Among other things, rough surfaces (632) can include micropores, metal, abrasive particles, dense particles or clumps of particles.

When the surgically created cavity dictates, first segment (662) is cylindrical and second segment (665) is conical.

When the surgically created cavity dictates, first segment (662) is biconvex and second segment (665) is conical. First segment (662) can be provided with a greater length than curved lengths of each opposed convex side (672a, 672b). First segment (662) can also be ovoid.

When the surgically created cavity dictates, first segment (662) is biconcave or concave.

As shown in FIG. 24, when the surgically created cavity dictates, patient-specific implant (650) can be provided with wings or bulges (670). Select preferred embodiments of patient-specific implant (650) shown in FIG. 14 can be provided with polyaxial head (100) connected to spheroid connector (102) that is attached to first cylindrical section (654). Polyaxial head (100) includes receptacle (110) that snap fits/locks over spheroid connector (102). As shown in FIG. 24, spheroid connector (102) can be offset of up to about 45 degrees from the longitudinal axis (X-X) of patient-specific implant (650).

FIG. 25 is a lateral perspective of patient-specific implant (690) including an exploded view of polyaxial head (100) and spheroid connector (102). First Region (616) can have a blunt tip (618) leading edge in front and connects posteriorly to second region (626p). The threadless friction fit surgical implant (690) in second region (626p) can have dimensions that maximize surface contact area with the geometric dimensions of the target zone. Second region (626p) is connected to first region (616) and third region (636p). Wings or bulges (670) can be incorporated into third region (636p). When medical parameters require, bends or curves can be incorporated into fourth region (646). Threadless friction fit surgical implant (690) can be provided with one or more openings (642) allowing interactions between conduit (624) and the target zone or surgically created environment proximate threadless friction fit surgical implant (690).

FIGS. 26a-26f show lateral perspectives of preferred embodiments of threadless friction fit surgical implant (690) for implantation into a patient. These preferred embodiments of threadless friction fit surgical implant (690) are provided with four distinct regions (616, 626p, 636p, 646). Third region (636p) can correspond to the area in between the pedicle and the mammillary process. Wings or bulges (670) can be incorporated into third region (636p). Bends or curves can be incorporated into fourth region (646). First region (616), second region (626p) and third region (636p) share a common midline (M-M). Fourth region (646) can share the common midline (M-M) or fourth region (646) can be provided with secondary midline (M1-M1) that is offset of up to about 45 degrees from the common midline (M-M).

Threadless friction fit surgical implant (690) is provided with a first region (616), a second region (626p), a third region (636p) and a fourth region (646) where the regions (616, 626p, 636p, 646) are interconnected. A preferred embodiment of threadless friction fit surgical implant (690) includes conduit (624) adapted to carry one or more biocompatible substances. Conduit (624) traverses first region (616), second region (626p) and third region (636p). Barrier (626) also surrounds conduit (624). In select preferred embodiments, conduit (624) can traverse fourth region (646) and barrier (626) surrounds fourth region (646).

Another preferred embodiment of patient-specific implant (690) includes an uninterrupted exterior (692). First region (616) includes a blunt tip (618). Outward dimensions of first region (616) are nearer to the conduit (624) than outward dimensions of the second region (626p) and third region (636p). Third region (636p) can be provided with one or more wings or bulges (637) uninterrupted exterior (692) where the wings (637) correspond to a target zone for implantation. Rough surfaces (632) can be applied to first region (616), second region (626p) and third region (636p). Fourth region (646), without rough surfaces (632), is connectable to a device distinct from threadless friction patient-specific implant (690).

FIGS. 27a-27f show lateral perspectives of preferred embodiments of patient-specific implants (690) shown in FIGS. 26a-26f that also include rough surfaces (632). Within the scope of the invention, there are an unlimited number of patient-specific spinal fixation devices (600) that can be manufactured by the current process (123). Examples of patient-specific spinal fixation devices (600) manufactured by the process (123) can include, but are not limited to, threaded conical and threadless porous ingrowth pedicle fixation devices.

### Preferred Steps of Process (123)

Depending on patient's (30) medical condition(s), metric output (408) of the current process can adjust manufacturing apparatus (500) to manufacture patient-specific implant(s) (600) that fit exactly into the target zone(s) (40), are slightly larger or smaller than target zone(s) (40) and/or add new additional structures associated with the three dimensional geometric representations (42) of the target zone (40). A user of the current process can utilize a visual display to adjust the metric output (408) before transfer to the manufacturing apparatus (500), e.g., structures can be added or deleted from manufactured patient-specific implant (600). For example, the metric output (408) correlated with patient's (30) X, Y and Z axes, three dimensional geometric representations (42) of the target zone (40) and volume of target zones (40) can cause manufacturing apparatus (500) to add such additional structures as apertures, overlaps, surface treatments or rough surfaces (632), and wings (637), etc. For select embodiments of the current invention, in view of the aggregate of medical histories, clinical observations and medical test results for multiple patients, the process (123) of making a patient-specific implant can suggest a patient-specific composition, volume, length, depth and/or additional structures of an implant that is tailored specifically for the patient having identical or similar medical history, test results and clinical observations.

Process (123) of making a patient-specific implant (series 600) for a patient (30) can include, but is not limited to, one or more scanners or scanning devices (100), communications devices (200), communications networks (300), computing devices, Cloud (570) computing or a combination thereof (400), aggregate (450) of encrypted and tagged information, manufacturing apparatus (500) and computerized navigation system (800). It is anticipated that communication networks (300) will interconnect the computing devices, Cloud (570) computing or a combination thereof (hereinafter computers (400) and scanners (100), communications devices (200), manufacturing apparatus (500) and computerized navigation system (800) that are situated at locations distinct from computers (400). However, the current invention can also function with scanning devices (100), communication networks (300), computers (400), manufacturing apparatus (500) and computerized navigation system (800) located at a centralized location such as a hospital.

FIGS. 1-6 portray representations of sagittal (X-axis), coronal (Y-axis) and transverse planes (Z-axis) of a portion of the spine. Patient (30) presents with a L4-5 spondylolisthesis. Medical scans (118) of target zones (40) were for some of the pedicles (902) of patent's (30) vertebra (920). As shown, target zones (40) of the pedicle (902) are round, oval and oblong. In select embodiments of the present invention, the aggregate of encrypted and tagged information (450) can generate a metric output (408) that causes the manufacturing apparatus (500) to make round, oval and oblong patient-specific implants (600) fit exactly into their target zone(s) (40) with only surface treatments (632) applied to first, second and third regions (616, 626p, 636p) of implant (600). If the surgeon's medical judgments were that different designs would improve safety during insertion or distribute postoperative stress loads over a greater surface area, the surgeon can use communication device (200) to adjust the metric output (408). By way of illustration, the surgeon's input can adjust the metric output (408) to cause manufacturing apparatus (500) to make the round patient-specific implant (600) to not have surface treatments (632) and be slightly larger than its target zone (40) and the oval patient-specific implant (600) to have no threads, have surface treatments (632) and fit exactly into its target zone (40).

Within the scope of the current process (123), the combination of memory (402), software (404) and processor (406) can publish reports. Reports 425 can be directed to the manufactured patient-specific implant (600) and target zone (40) and include, strength, dimensions, required implantation force, visual three-dimensional representations (42), customizable options, risk of iatrogenic fracture, etc. Reports (426) can be associated with the outcomes of patient-specific implants (600) and can assist in patient care, hospital efficiency, and manufacturing. Educational reports (427) can compare different adjunct medical treatments of patients receiving patient-specific implants (600) made by the current process (123) can be generated.

Select Embodiments of Hardware and Software Associated with Process (123) Disclosed in FIGS. 28-30.

### First Preferred Embodiment of the Process (123)

Scanners (100) are adapted to intercommunicate with one or more communications devices (200). Communication devices (200) can be integral with scanners (100) or distinct from scanners (100), and within the scope of the current process of making a patient-specific implant (600) for a patient (30), communications between scanners (100) and one or more communications devices (200) can be bidirectional.

In accordance with the current process (123), scanners or scanning devices (100) utilize one or more scanning techniques selected from the group consisting of X-rays, computerized tomography (CT), magnetic resonance imaging (MRI) and dual-energy X-ray absorptiometry (DEXA), ultrasound, or any other scanning technique acceptable in the art that can be utilized to scan target zone (40).

Communications devices (200) compatible with the current process of making a patient-specific implant (600) for a patient (30) can include desktop computers, tablet computers, smart phones or any other device, acceptable in the art, capable of encrypting, sending and receiving data. Preferred communications devices (200) can be provided with communications processor (202), store (204) and visual display (206). Hereinafter, “communications processor” (202) refers to any processor associated with a communications device (200) and “store” (204) refers to any memory associated with a communications device (200).

Communications devices (200) can intercommunicate with each other and one or more communications networks (300) that can transfer bidirectional communications between communications devices (200) and computers (400).

For preferred embodiments, instead of using a network (300), one or more communications devices (200) can intercommunicate directly with one of computers (400). Other select preferred embodiments of communications devices (200) that include software (404) can intercommunicate directly with manufacturing apparatus (500). Among other things, computers (400) are provided with memory (402), software (404) and processor (406). Processor (406) and software (404) calculate metric output (408) utilized by manufacturing apparatus (500) to make patient-specific implant (600). For selected computers (400), memory (402) can include RAM and ROM.

Depending on preselected parameters, computers (400) can intercommunicate with manufacturing apparatus (500) and deliver software (404) calculated metric output (408) to manufacturing apparatus (500). Metric output (408) directs manufacturing apparatus (500) to manufacture patient-specific implant (600) for patient (30) according to the specifications of metric output (408). Communications device (200) can be used to adjust metric output (408) before transfer to the manufacturing apparatus (500).

A combination of one or more of three dimensional geometric representations (42) of the current patient's (30) target zone (40) and the selected metric output (408) for patient-specific implant (600) can be communicated to a computerized navigation system (800) in an operating room (840) for assisting with the implantation of patient-specific implant (600) into the patient's (30) target zone (40).

With respect to data encryption associated with the process of making a patient-specific implant (600) for a patient (30), Hypertext Transfer Protocol Secure (HTTPS) or another protocol acceptable in the art can be utilized to encrypt data transmissions between communications devices (200), networks (300), computers (400) and manufacturing apparatus (500). After any data/information is entered into the current process of making a patient-specific implant (600) for a patient (30), one or more communication protocols secure the encrypted data/information in regards to any Cloud (570) or Non-Cloud communications associated with preoperative, operative, postoperative, manufacturing, navigational, scanning or computing communication/intercommunication associated with the present invention.

### Second Embodiment of the Process (123)

Communications networks (300), computers (400) are adapted to communicate with each other. Among other things, computers (400) include processors (406), memory (402) and software (404). Memory (402) is adapted to securely contain an aggregate (450) of encrypted and tagged information correlated with patient-specific implants (600) previously implanted into prior patients (30) and manufactured by the process of making a patient-specific implant (600) for a current patent (30).

Aggregate (450) of encrypted and tagged information can include one or more of the following: correlation of medical scans (118) and locations of the target zones (40) with X, Y and Z axes of the prior patients (30); the target zones (40); three-dimensional geometric representations (42), with or without implants (600), of the prior patients' (30) target zones (40); prior patients' (30) medical histories, clinical observations and medical test results; prior patients' (30) metric outputs (408) for the dimensions of prior patients'patient-specific implants (600); and/or a registry of government approved safe and effective implants available for implantation into patients.

In select preferred embodiments of the process of making a patient-specific implant (600) for a current patient (30), preoperative, operative and postoperative medical histories, medical conditions, clinical observations, medical tests and/or surgical outcomes of patients (30) previously receiving patient-specific implants (60) are added into the aggregate (450) of encrypted and tagged information. In other preferred embodiments of the process of making a patient-specific implant (600) for a current patient (30), aggregate (450) can include such information as identities of surgeons, physicians and medical facilities and dates where patient-specific implants (600) were implanted into patients (30).

Processor (406) and software (404) can be programmed to accumulate additional predetermined information for aggregate (450). In accordance with the current invention, the combination of processor (406) and software (404) provide search capabilities of the aggregate (450) of encrypted and tagged information.

Scanners (100) are adapted to intercommunicate with one or more communications devices (200). Communication devices (200) can be integral with scanners (100) or distinct from scanners (100). Within the scope of the current process of making a patient-specific implant (600) for a patient (30), communications between scanners (100) and one or more communications devices (200) can be bidirectional.

In accordance with the current process (123) of making a patient-specific implant (600) for a patient (30), one or more scanners (100) identified above and are utilized to scan target zone (40).

Communications devices (200) can intercommunicate with each other and one or more communications networks (300) that can transfer bidirectional communications between communications devices (200) and computers (400). Networks (300) compatible with the current process (123) of making a patient-specific implant (600) for a patient (30) include radio frequency, the Internet, the PTSN, a LAN, a WAN, VoIP, wired communications, wireless communications, any combination thereof or any other network acceptable in the art.

For select preferred embodiments, instead of using network (300), one or more communications devices (200) can intercommunicate directly with one of the computers (400). Other select preferred embodiments of communications devices (200) that include software (404) can intercommunicate directly with manufacturing apparatus (500). Among other things, computers (400) are provided with memory (402), software (404) and processor (406). Processor (406) and software (404) calculate metric output (408) utilized by manufacturing apparatus (500) to make patient-specific implant (600). Memory (402) can include RAM and ROM.

Depending on preselected parameters, computers (400) can intercommunicate with manufacturing apparatus (500) and deliver software (404) calculated metric output (408) to manufacturing apparatus (500). Metric output (408) directs manufacturing apparatus (500) to manufacture patient-specific implant (600) for patient (30) according to the specifications of metric output (408). In select preferred embodiments of the process of making a patient-specific implant (600) for a patient (30), metric output (408) can be adjusted before transfer to manufacturing apparatus (500).

Among other things, computers (400) correlate medical scans (118) from the various diverse scanners' (100) scans (102) of the locations the target zones (40) with X, Y and Z axes of the patient (30). This correlation of data with X, Y and Z axes of the patient (30) allows software (400) and computers (400) to create viewable three dimensional geometric representations (42) of the current patient's (30) target zone (40) and generate metric output (408). Select embodiments of the present process can calculate the X, Y and Z axes relative to a specific vertebral body (901) to further sub-classify aggregate (450) and improve overall search precision of aggregate (450). For select preferred embodiments, a combination of one or more of three dimensional geometric representations (42) of the current patient's (30) target zone (40) and the selected metric output (408) for patient-specific spinal fixation device (600) can be communicated to a computerized navigation system (800) in an operating room (840) for assisting with the implantation of patient-specific spinal fixation device (600) into the patient's (30) target zone (40).

Within the scope of the current invention, manufacturing apparatus (500) can utilize one or more of the following methodologies of acid etching, milling, molding, printing, 3-D printing, welding, or any other methodology acceptable in the art, to make the patient-specific implant (600).

Software (404) can cause the calculated metric output (408) to add surface treatments or roughness (632) to patient-specific implant (600). Surface treatments (632) can be applied to one or more sections of patient-specific implant (600) to increase frictional resistance to movement and facilitate bone ingrowth. Manufacturing apparatus (500) can be utilized to create micropores (632) and/or barbs, etc. on surfaces of patient-specific implant (600). Surface treatments (632) can be created by multilayer printing, abrasive devices, chemical, laser, metal or abrasive particles incorporated into or onto the patient-specific implant (600) or by other means acceptable in the art. Optionally, software (404) can cause manufacturing apparatus (500) to manufacture a model of target zone 40.

When medical conditions require, software (404) causes the calculated metric output (408) to add additional structures to patient-specific implant (600). Examples of additional structures include but are not limited to: apertures, cannulas, fenestrations, meshes, ridges, threads and wings (637). In the alternative, when medical engineering parameters require, software (404) causes the calculated metric output (408) to create a patient-specific implant (600) with smooth external surfaces.

### The Computerized Method of Making Orthopedic Reconstructions (123A, 123B, 123C)

FIGS. 31-53A provide depictions of orthopedic reconstructions (600R) and the computerized methods (123A-C) available to manufacture orthopedic reconstructions (600R) as well as the patient-specific implants (600) previously portrayed in FIGS. 1-30.

Optimal computerized methods (123A-C) can utilize one or more scanners (100), communications devices (200), networks (300), computers (400), aggregate (450) of encrypted and tagged information, manufacturing apparatus (500) and computerized navigation system (800).

Optimal methods (123A-C) of making an optimal patient-specific orthopedic reconstruction (600R) for a target zone (40) within or on the patient can be achieved through, among other things, advanced data aggregation, prognostic scoring, and metric output optimization. The current optimal methods (123A-C) use encryption, decryption and tagged information to maintain patient privacy and are compliant with the necessary regulatory bodies, such as the Health Insurance Portability and Accountability Act (HIPAA), the US Center for Medicare and Medicaid Services, the US Food & Drug and Administration or any other similar worldwide jurisdictional regulatory body. Tags clearly mark the data as sensitive protected health information causing computing apparatus (480) to enforce specific, legally required security protocols, access controls, and retention policies on data during its sending and storage. Computing apparatus (480) can include servers, storage arrays, networking equipment, hypervisor, virtual machines, containers, operating systems, Cloud (570) services, management and automation tools, client devices (200), e.g., desktop and mobile devices, web browsers, programs, apps and management portals.

As utilized in this Application, tagging (tags) can apply tags to raw data, e.g., patient-specific digitized medical scans (118), metadata and other medical data. Tags can include details such as patient identify to a specific record (often a unique, anonymized ID), data type source (CT scan, MRI, target zone (40), security level protected health information), coordinate data related to the relative X, Y, Z axes associated with target zone (40). Tags are needed for searchability and retrievability when using storage array (484) as well as linking medical data with a specific patient. As related to target zone (40), metadata tags can include bibliographic data, patent numbers, grant date, filing date, inventor names, assignee/company, IP classifications, legal events, governmental regulations, owner as well as information regarding how data is stored. In short, “tagging” is a technique used by computerized methods (123A, 123B, 123C) to organize, identify, and categorize the medical data, which enables the remaining claimed steps of “sending and securely storing” the correct, properly identified information.

### Optimal Patient-Specific Structural Configurations

Within the scope of the current invention are optimal patient-specific orthopedic reconstructions (600R) which can include any traditional bone implants (embodiments (600, 620, 650, 690) disclosed previously, other bone implants not previously disclosed, bone replacement segments and complete bone replacements (600R) embodied hereafter. Orthopedic reconstructions (600R) include can include but not limited to the following bones: acetabulofemoral joint, antebrachium, cranium, femur, foot, manus, humerus, knee, mandible, maxilla, scapula, thoracic skeleton, tibia, vertebrae or boney portions thereof, elbow and bionic hand. Implants, segmental replacements, fixation devices, dental/craniofacial devices and bionic hands can also be manufactured by methods (123A-C). Computations of the patient-specific orthopedic reconstruction for any specific patient's target zone (40) are associated with any preselected bone's X, Y, Z axes, patient's X, Y, Z axes relative to the geometric center of the patient's body mass or other predetermined location of a patient's mass or a combination thereof. Additionally, the current invention can also manufacture the customed-fitted orthopedic reconstructions (600R) with distinct functional regions and structural applications and treatments, such as those previously identified with process (123).

Among other things, optimal customed-fitted orthopedic reconstructions (600R) can also include heads, polyaxial heads (100), tips (618), shafts, blunt ends, sharp ends, cutting edges, threads, porous ingrowth surfaces (638p), cages, internal conduits (624), solid masses, openings, different regions for different functions, resilience, bulges (670), serrations (635), wings (637), ridges or a threadless friction fit and depending on the target zone (40) are preferably manufactured with porous ingrowth surfaces (638p), rough surfaces (632) or any combination thereof. It is anticipated that most of the optimal patient-specific orthopedic reconstructions (600R) will be manufactured to facilitate osseointegration.

The Computerized Method of Making a Patient-Specific Orthopedic Reconstruction (123A-C) is adapted to optimize the orthopedic reconstruction's design structure beyond a simple geometric fit to improve long-term clinical success.

### Data Processing and Metrics Calculation—Generating a Patient-Specific Model (600M)

Computing apparatus (480) activates software (420) to process the digitized medical scans (118) to create a patient-specific three-dimensional model (600M) of target zone (40). Examples of medical scanners (100) include, but are not limited to X-rays, ultrasound, computerized tomography (CT), magnetic resonance imaging (MRI), dual-energy X-ray absorptiometry (DEXA) and any other state-of-the art scanner. Relative to the patient's predetermined X, Y, Z axes, one or more three-dimensional models (600M) associated with the target zone (40) are generated—producing one or more patient-specific three-dimensional images of orthopedic reconstruction (600R) that can be implanted precisely into the current patient's designated surgical field. In accordance with the current invention, video representations of target zones (40), three-dimensional models (600M), three-dimensional orthopedic reconstructions (600R), operative metrics and suggestions as well as surgical robot (820) control are visible on one or more displays (900) remote from computing apparatus (480). Among other things, computing apparatus (480) can utilize and access software (420) including software such as SaaS and other computing devices. Computing apparatus (480) can also include virtual servers (482), storage array (484), real time communications with communications devices (200), manufacturing devices (500), surgical robot (820) and audiovisual displays (206) in the surgical suite (840) and other locations remote from the surgical suite (840).

### Calculating the First Set of Metrics—Computerized Method (123A)

In select preferred embodiments, computing apparatus (480) compares the current patient's three-dimensional model (600M) and current patient-specific medical data with the aggregate (450) of historical implant and outcome data from prior patients. A first set of patient-specific metrics (460) is calculated based on the current patient's three-dimensional model (600M). The first set of patient-specific metrics (460) define the implant's basic fit parameters, such as length, width, taper, and angulation for the current patient's anatomy.

Select embodiments of the patient's three-dimensional model (600M) can include a first second and a second section opposed to the first section where the second section is tapered such that the cross-sectional area of the optimal patient-specific three-dimensional model (600M) is less proximate the patient's target zone (40). The current patient's three-dimensional model (600M) can create a metric (470) for fabricating the optimal patient-specific orthopedic three-dimensional reconstruction (600R) that can generate simultaneously a conduit (624) extending through the optimal patient-specific orthopedic three-dimensional reconstruction (600R) about an axis of the optimal patient-specific orthopedic three-dimensional reconstruction (600R) during a 3D printing process (500). The patient's three-dimensional model (600M) can generate an optimal patient-specific orthopedic three-dimensional reconstruction (600R) including bulges (670), serrations (635) and/or wings (637) that are computationally optimized to inhibit over-insertion of the optimal patient-specific three dimensional reconstruction (600R), increase the surface area for bone ingrowth, dissipate loads transmitted from adjacent levels proximate the target zone (40) and a connector for coupling devices distinct from the optimal patient-specific orthopedic three dimensional reconstruction (600R). An algorithm is adapted to create a metric (470) for fabricating the optimal patient-specific three-dimensional orthopedic reconstruction (600R) to create a non-uniform, patient-specific structural lattice that provides a greater load-bearing capacity and a reduced risk of stress shielding. Computerized method (123A) can be used to manufacture patient-specific implant (600).

### Aggregate and Prognostic Scoring—The Computerized Method (123B)

An Osseointegration Probability Score (OPS) (123Q) can be calculated by comparing the patient-specific implant model (600M) with a three-dimensional template (600T) of the surgical anatomy associated with target zone (40) and the aggregate (450) data calculated by computerized method (123A) and stored on storage array (484). Computing apparatus (480) executes software (420) to compare and generate advanced prognostic scores to calculate an OPS (123Q). OPS (123Q) and utilize data including a surgical plan, bone mineral density (BMD) calculation, preselected manufacturing substances, and biomechanical load simulations predictions. Importantly, OPS (123Q) can also predict the likelihood of successful fusion and bone ingrowth.

### Comprehensive Prognostic Success Score—The Computerized Method (123C)

The Comprehensive Prognostic Success Score (CPSS) (123S) (expands on and includes OPS (123Q) to predict the likelihood of overall longer-term implant success. Computing apparatus (480) and software (420) calculate CPSS (123P) factors such as the likelihood of successful osseointegration, infection resistance, material wear characteristics, patient quality-of-life indications and patient gait analysis. Among other things, quality-of-life indications can include the current patient's physical health and functioning, mobility, symptoms, discomfort, emotional status, cognitive capabilities, self-perception, social functioning, role functioning and spirituality.

### Calculation of the Second Set of Optimized Metrics

The second set of optimized metric outputs (490) is calculated by analyzing the first sets of metrics (460) in view of either of the prognostic scores (OPS/CPSS). Computing apparatus (480) and software (420) actively utilize the predicted outcomes to suggest modifications that improve safety and performance of the three-dimensional patient-specific orthopedic reconstruction (600R). An optimal metric (490) is selected from the first set, the second set, or a combination thereof, and is transmitted to the manufacturing apparatus (500) for fabrication of the three-dimensional patient-specific orthopedic reconstruction (600R). Within the scope of the current invention, the surgeon has the ability to override the calculated optimal metric output (490) before transmission to manufacturing apparatus (500).

### Advanced Computational Optimization—Computerized Methods (123B, 123C)

Computerized methods (123B, 123C) can be utilized to manufacture patient specific implants (600) and patient-specific three-dimensional reconstructions (600R).

To improve fit and performance, select preferred embodiments of software (420) can be adapted to employ sophisticated computational methods. Using machine learning/deep learning, software (420) can employ a deep learning neural network or other machine learning model trained on the aggregate historical data (450) to calculate the CPSS (123S) and OPS (123Q). Furthermore, the software (420) can execute a biomechanical load simulation, preferably using a finite element analysis model which incorporates patient-specific bone mineral density (BMD) data and predicted muscle force vectors derived from the medical scans (118). Software (420) can also create smart bionics (600RN). An optimal metric output (490S) transmitted to the manufacturing apparatus (500) can comprise a calculated non-uniform porous lattice density map. The map is dynamically varied across the orthopedic reconstruction's volume based on the calculated biomechanical load simulation and the CPSS (123S). Via 3D printing, the optimal patient-specific orthopedic reconstruction to be manufactured with varying internal densities to reduce stress shielding while maintaining high load-bearing capacity.

Structural elements and features previously described, such as bulges (670), serrations (635), and/or wings (637) and smart bionics (600RN), are computationally optimized (e.g., dynamically modified by an algorithm) in response to the optimal metric (490S) to perform specific functions, including, but not limited to: inhibiting over-insertion of the patient-specific orthopedic reconstruction (600R), increasing the surface area for bone ingrowth, dissipating loads transmitted from adjacent spinal levels proximate the target zone (40), bionic joint movements.

### Computerized Method (123B) for Calculating the Osseointegration Probability Score (123Q)

The Osseointegration Probability Score (OPS) (123Q) utilizes advanced computing and modeling to move beyond standard surgical planning toward a personalized prediction of biological outcome. Patient-specific three-dimensional model (600M) is a virtual representation of the optimal patient-specific reconstruction (600R) architecture in view of target zone (40). Patient-specific three-dimensional model (600M) is calculated in view of target zone (40), aggregate (450) potential patient-patient-specific data regarding density, structures, compositions and compatibility with target zone (40), among other things. The patient-specific three-dimensional template (600T) is the virtual representation of the final, desired anatomical outcome. It serves as a geometric and structural ideal which is compared with the patient-specific three-dimensional model (600M).

Aggregate (450) is a comprehensive, multi-factorial data set providing current patient-specific and prior patients' biological, mechanical and historical contexts needed to assess the environment where optimal patient-specific three-dimensional reconstruction (600R) will be positioned. See FIG. 56 regarding examples of holistic components utilized by method (123B) to calculate OPS (123Q).

OPS (123Q) is the quantitative output of the comparison algorithm. The algorithm processes the input data for the patient-specific three-dimensional model (600M) and weights the factors from the aggregate (450) according to known impact on bone-implant fixation, e.g., high stress shielding in a target zone (40) of low bone density would handicap OPS (123Q). The resulting OPS (123Q) is a single or multi-dimensional numerical value (e.g., a score from 0 to 100, or a probability percentage) predicting likelihood of successful long-term osseointegration for manufactured optimal patient-specific three-dimensional reconstruction (600R).

OPS (123Q) calculations by computerized method (123B) can advantageously:

Provide surgeons and engineers ability modify optimal patient-specific three-dimensional reconstruction (600R) by utilizing the patient-specific three-dimensional model (600M) to immediately recalculate OPS (123Q). For example, this allows informed changes to optimal patient-specific three-dimensional reconstruction (600R), e.g., adjusting lattice density until a maximum OPS (123Q) is achieved, effectively minimizing the risk of failure before surgery.

Allow clinicians to quantitatively assess and communicate the risks associated with optimal patient-specific three-dimensional reconstruction (600R).

Serve as prognostic indicator of long-term implant survival and biological success.

A machine learning model trained on the aggregate data (450) can be an artificial intelligence (AI) algorithm that has been statistically optimized using a massive, diverse dataset to perform a specific prediction task such as calculating OPS (123Q). The algorithm can be a neural network, a decision tree, or a random forest learning complex patterns and relationships without being explicitly programmed to do such tasks. Data from thousands of previous patients and operations for similar implant/reconstruction procedures is calculated. Among other things, the dataset can include patients'anatomies, medical histories, biomechanical simulations and replications, surgical plans, or other data crucially linked to final successful or failed outcomes for the patients including indications regarding success or failure. Such accumulated data sets allow computing apparatus (480), software (420) and machine learning to calculate OPS (123Q) and predict the probability of success or failure for optimal patient-specific three-dimensional reconstruction (600R) associated with target zone (40).

### Computerized Method (123C) for Calculating the Comprehensive Prognostic Success Score (CPSS) (123S)

Computerized Method (123C) introduces a method for generating a Comprehensive Prognostic Success Score (CPSS) (123S) which represents an ultimate, holistic, and long-term prediction of the functional and biological success of the optimal patient specific three-dimensional reconstruction (600R). Among other things, CPSS (123Q) builds upon and incorporates OPS (123Q). Method (123C) and CPSS (123S) extend the scope of the current invention beyond method (123B) to include additional comprehensive assessments of clinical and engineering variables.

CPSS (123S) is calculated by executing a sophisticated machine learning model on the computing apparatus (480). CPSS (123S) compares patient-specific three-dimensional model (600M) against aggregate (450) of historical and other patient data including multi-domain data from prior patients and as well as long-term outcomes. For example, failures due to infection or wear and successful ten plus or twenty plus years survival for optimal patient-specific three-dimensional reconstructions (600R) associated with target zone (40). CPSS (123S) can also determine which engineering parameters for each type of patient-specific three-dimensional reconstructions (600R) were better. See FIG. 77 regarding examples of holistic components utilized by method (123C) to calculate OPS (123S).

CPSS (123S) is a single quantitative score or a vector of weighted sub-scores predicting the likelihood of overall success of patient-specific three-dimensional reconstructions (600R). Over time, it is anticipated that CPSS (123S) scores will be incorporated into most medical surgical procedures.

### Comprehensive Success Determination

Aggregate (450), software (420) and computing apparatus (480) and computerized method (123C) improve on computerized method (123B). Along with calculating OPS (123Q), CPSS (123S) also determines the following:

Infection Resistance: predicting the likelihood of patient-specific three-dimensional reconstructions (600R) periprosthetic complications associated with infection, physical components and structures, geometry related to corresponding target zone, (which affects fluid pocketing), and the patient's specific infection markers identified in aggregate (450). Examples of specific infection markers include, but are not limited to systemic inflammatory markers, localized markers, culture and genetic markers and patient risk factors.

Composition Wear Characteristics: Forecasting the long-term performance of materials (e.g., polymer liners, ceramic surfaces, biocompatible metals, graphene) based on aggregate's (450) generated based on calculated biomechanical loads and historical failure rates for previous and similar patient-specific three-dimensional reconstructions (600R) with similar medical histories.

Patient quality-of-life (QOL) indications: predicting functional outcomes, pain reduction, and mobility restoration based on the optimized fit and historical patient self-reported outcomes.

### Manufacturing, Implantation, Attachment

Manufacturing apparatus (500) can be any state-of-the art apparatus capable of making the optimal patient-specific three-dimensional orthopedic reconstruction (600R). However, it anticipated that a 3D printing device (500) will currently be the more common selection to fabricate the optimal patient-specific three-dimensional orthopedic reconstruction (600R). Optimal metric (490, 490S) can cause 3D printing device to simultaneously generate a conduit (624) spanning an internal length of the optimal patient-specific orthopedic three-dimensional reconstruction (600R), a non-uniform porous lattice density, apertures extending inward to the conduit from the orthopedic reconstruction's (600R) exterior, roughness, smoothness, additive/subtractive physical structures including self-sustaining bionics, dry circuits, standard circuits, sensors, moving joints, conductive filaments, conductive inks, tension wires and microcontrollers.

In accordance with the current methods (123A-123C), examples of substances that can be used for the manufacture of the optimal patient-specific orthopedic reconstruction (600R) implant include, but are not limited to: Titanium and titanium alloys, cobalt-chromium alloys, graphene, stainless steel, ultra-high molecular weight polyethylene, polyetheretherketone, polymethyl methacrylate, biodegradable polymers, ceramics, diamonds, hydroxyapatite, tricalcium phosphate, alumina, zirconia, scaffolds holding autographs, allographs, xenographs or any combination thereof.

The current computerized methods for making a patient-specific orthopedic reconstruction (123A, 123B, 123C) create an optimal reconstruction (600R) specifically tailored to the current patient's anatomy, medical history, and biomechanical needs, thereby increasing the probability of a successful long-term outcome. Computerized methods (123A, 123B, 123C) can also transmit data to a computerized navigation system (800) to assist a surgeon's operative implantation into the patient's target zone (40) corresponding with the patient's predetermined surgical field. All stored data and transmissions associated with the current invention are encrypted, decrypted, tagged and transmitted in accordance with the Health Insurance Portability and Accountability Act (HIPAA) or other jurisdictional regulatory bodies laws and regulations requiring secure data storage and transmissions.

Methods (123A), (123B) and (123C) can utilize a combination of Generative AI for design and Discriminative/Analytical AI for processing, quality assurance, and control.

Generative AI is focused on creating new, optimal reconstructions (600R) and 3D models (600M) based on specific patient data and performance constraints. Generative AI can use Deep Learning models such as Generative Adversarial Networks or Variational Autoencoders. Algorithms explore thousands of design possibilities for reconstruction (600R) based on user-defined medical parameters. Generative AI can automatically create lightweight, high-strength, and anatomically perfect lattice structures that are impossible for humans to design manually. This is crucial for optimizing reconstruction (600R) structures for production via additive manufacturing (500), e.g., 3D printing.

Analytical AI focuses on interpreting patient data, ensuring the quality of the final product, and controlling the device's function. Medical image segmentation can utilize deep learning convolutional neural networks, especially U-Nets. By way of illustration, convolution neural networks can create a model that automatically analyzes 2D slices from patient scans (102) and partitions the image to isolate and define the precise boundaries of the anatomical structure relative to target zone (40). From the original raw medical scan's data (102), convolution neural networks create a clean, 3D digital model (600R) that significantly reduces time and manual effort required by engineers to design the optimal patient-specific orthopedic three-dimensional reconstruction (600R).

Quality Control & Defect Detection can utilize computer vision (a subset of Deep Learning, mainly using convolutional neural networks). AI-powered cameras and sensors monitor the manufacturing process (500) in real-time. Models (600M) are trained on images of perfect parts to quickly identify microscopic defects, porosities, or surface irregularities of optimal reconstruction (600R). Quality Control & Defect Detection ensures the final optimal reconstruction (600R) meets stringent medical safety standards, especially for high-precision techniques like metal 3D printing. AI inspection is faster than manual review of the manufactured optimal reconstruction (600R).

Process Optimization & Predictive Maintenance can utilize machine learning (e.g., time series regression models (600M). Time series regression models (600M) analyze real-time data (temperature, laser power, vibrations, etc.) from 3D printers or manufacturing apparatus (500) to predict potential equipment failures before they occur. This can reduce costly downtime, minimizes material waste from failed reconstructions, and improves consistency of the entire manufacturing process.

Manufacture of prosthetic control systems can use machine learning such as recurrent neural networks or long short-term memory networks. For advanced bionic prosthetics, these algorithms interpret complex, sequential electrical signals from the patient's muscles (EMG signals) or nerves (neural interfaces). The algorithms can translate the user's mental intention into real-time, fluid resulting in intuitive movements of bionic reconstruction (600R). This provides the patient with a more natural and personalized experience when using the bionic reconstruction (600R).

The Figures identified below represent specific examples of optimal patient-specific three-dimensional reconstructions (600R) manufactured by computerized methods of making a patient-specific orthodontic reconstruction (123A, 123B or 123C), without limiting the scope of methods (123A, 123B, 123C). Instead, the Figures portray numerous target zones (40). Methods (123B, 123C) can be used to manufacture an optimal reconstruction (600R) for virtually any bone or joint as well as external medical devices utilized for treatment of target zone (40).

FIG. 31 illustrates shattered ulna (1010), target zone (40) an optimal orthopedic reconstruction (600RA) of a complete bone, manufactured in accordance with method (123A), (123B) or (123C). The optimal orthopedic reconstruction (600RA) is manufactured according to a physician directed surgical plan to best meet the patient's needs and includes features similar to a normal ulna.

FIG. 32 illustrates bone segment (1020) of a cranium, target zone (40) and an optimal orthopedic reconstruction (600RB) manufactured in accordance with method (123A), (123B) or (123C). The optimal orthopedic reconstruction (600RB) is manufactured according to a physician directed surgical plan to best meet the patient's needs. The reconstruction fits precisely into the target zone (40) to protect the brain and includes textures that feel like a healthy skull and also includes the necessary support for artificial hair replacement or natural hair regrowth.

FIG. 33 illustrates a humerus (1030), target zone (40) and an optimal orthopedic patient-specific reconstruction (600RC) manufactured in accordance with method (123A), (123B) or (123C). The optimal orthopedic patient-specific reconstruction (600RC) is manufactured according to a physician directed surgical plan to best meet the patient's needs. Reconstruction (600RC) fits precisely into target zone (40) of section (1032) where injured natural bone was removed from humerus (1030). Reconstruction (600RC) can be manufactured to include the inward and outward appearance of the patient's remaining healthy humerus (1030).

FIG. 34 illustrates a femur (1040), target zone (40) and an optimal orthopedic reconstruction (600RD) manufactured in accordance with method (123A), (123B) or (123C) that can be positioned inside the medullary cavity of femur (1040). An optimal orthopedic patient-specific reconstruction (600RD), according to a physician directed surgical plan to best meet the patient's needs, is manufactured. Among other things, reconstruction (600RD) can include the outward appearance of the patient's natural bone as well as longitudinal ends (600RD1, 600RD2) that correspond precisely with the contacted natural bone (1040).

FIG. 35 illustrates a multi-component optimal orthopedic reconstruction (600RE) and target zone (40) for a knee (1050) including femur component (1052), tray (1054) and insert (1056) manufactured in accordance with method (123A), (123B) or (123C). Multi-component optimal orthopedic reconstruction (600RE) is geometrically sized and shaped to represent a normal knee joint for the specific patient's body mass.

FIG. 36 illustrates a foot (1060) with an optimal orthopedic reconstruction (600RF) and target zone (40), e.g., calcaneus plate (600RF) manufactured in accordance with method (123A), (123B) or (123C). According to methods (123A), (123B) or (123C), the patient-specific calcaneus reconstruction (600RF) can be manufactured to precisely fit with the patient's remaining natural foot bones.

FIG. 37 illustrates a target zone (40) and a patient-specific optimal orthopedic reconstruction (600RG), e.g., eternal fixator (600RG) for the femur and tibia, manufactured in accordance with method (123A), (123B) or (123C). In accordance with method (123A), (123B) or (123C) patient's body mass and other specifications calculations, patient-specific optimal orthopedic reconstruction (600RG) components are manufactured to precisely interact with the patient's femur (1040) and tibia (1042).

FIG. 38 illustrates a mandible (1070) with a missing tooth target zone (40).

FIG. 38A illustrates a patient-specific optimal orthopedic reconstruction (600RH) for implantation into target zone (40) manufactured in accordance with method (123A), (123B) or (123C). Within the scope of methods (123A), (123B) or (123C), any number of patient-specific optimal orthopedic reconstructions (600RH) preselected target zones (40) or complete mandible and maxilla teeth replacements (600RH) can be precisely manufactured and implanted into the patient.

FIG. 39 illustrates a vertebra (1090) including an exploded view of a thoracic section (1090A) with target zone (40) for receiving implantation of the optimal orthopedic reconstruction (600RI), e.g., a precisely engineered pedicle screw manufactured in accordance with method (123A), (123B) or (123C), for implantation into target zone (40).

FIG. 40 illustrates the acetabulofemoral joint (1110) and target zone (40).

FIG. 40A illustrates an optimal orthopedic reconstruction's (600RJ) components (600RJ1, 600RJ2, 600RJ3, 600RJ4) for implantation into target zone (40) manufactured in accordance with method (123A), (123B) or (123C).

FIG. 41 illustrates the humerus-scapula joint (1120) and target zone (40).

FIG. 41A illustrates optimal orthopedic reconstruction's (600RK) components (600RK4, 600RK3, 600RK2, 600RK1) for implantation into target zone (40) manufactured in accordance with method (123A), (123B) or (123C).

FIG. 42 illustrates a thoracic skeleton (1130), a clavicle (1130A) and target zone (40).

FIG. 42A illustrates optimal orthopedic reconstruction′ (600RL) components (600RL1, 600RL2) manufactured in accordance with method (123A), (123B) or (123C) for use with target zone (40) of clavicle (1130A).

FIG. 43 illustrates an elbow (1140) target zone (40). Optimal orthopedic reconstruction (600RM) is manufactured in accordance with method (123A), (123B) or (123C) and the selected method generates interactive components (600RM1, 600RM2, 600RM3) creating artificial replacement parts for elbow (1140).

FIG. 44 illustrates an optimal orthopedic reconstruction (600RN) of bionic hand (1152), movable thumb (1153) and fingers (1154), circuity (1555), tension wires (1156), sensors (1157), energy store (1158) and microcontroller (1159) including store firmware (1160). Method (123A), (123B) or (123C) can be utilized to manufacture optimal orthopedic reconstruction (600RN) including connectors for precise attachment to the patient's arm.

FIG. 45 illustrates an orthopedic reconstruction (600RO) for a portion (1170F) of a femur (1040). Reconstruction (600RO) is manufactured within the scope of Computerized Method of Making a Patient-Specific Orthopedic Reconstruction (123A, 123B, 123C) enabled herein.

FIG. 45A portrays a cross-section of orthopedic reconstruction (600RO) including a first porous lattice (1172F).

FIG. 45B shows a second porous lattice (1172S) for orthopedic reconstruction (600RO).

FIG. 46 illustrates thoracic vertebra (1180V) with a defect at target zone (40) an orthopedic reconstruction (600RP) that is a regenerative scaffold. Optimal orthopedic reconstruction (600RP) is manufactured within the scope of Computerized Method of Making a Patient-Specific Orthopedic Reconstruction (123A, 123B or 123C) enabled herein.

FIGS. 47-47C illustrate orthopedic reconstructions (600RQ, 600RQ1, 600RQ2, 600RQ3) that are distinct regenerative meshes (1190, 1190A, 1190B, 1190C) manufactured by a computerized method (123A, 123B, 123C).

FIGS. 48-48B illustrate orthopedic reconstructions (600RR, 600RR1, 600RR2) that are distinct nanogenerators (1200, 1200A, 1200B) manufactured by a computerized method (123A, 123B, 123C).

FIGS. 49-49B illustrate orthopedic reconstructions (600RS1, 600RS2, 600RS3) that are distinct biometric sensors (1202, 1202A, 1202B, 1202C) manufactured by a computerized method (123A, 123B, 123C).

FIG. 50 illustrates orthopedic reconstruction (600RT) incorporating diverse densities for the benefit of the patient manufactured by a computerized method (123A, 123B, 123C). By way of illustration, diverse densities can include cortical (1210), gradient (1212) and cancellous (1214) densities.

FIG. 51 illustrates orthopedic reconstruction (600RS) manufactured from a metal (600RS1), metal-polymer combination (600RS2) and a polymer (600RS3) manufactured by a computerized method (123A, 123B, 123C).

FIG. 52 illustrates orthopedic reconstruction (600RT) with differing lattice densities manufactured by a computerized method (123A, 123B, 123C).

FIG. 53 is a flow diagram illustrating methodologies of the Computerized Methods of Making a Patient-Specific Orthopedic Reconstruction (123A, 123B, 123C).

FIG. 53A portrays remote settings and devices inter communicating with Cloud (570) and computing apparatus (480).

FIGS. 54-73 disclose computerized methods (123A, 123B, 123C) of making a patient-specific orthopedic reconstruction.

With reference to FIGS. (53-53A), computing apparatus (480) resides in Cloud (570). Computing apparatus (480) includes virtual server (482), non-transitory memory (402), software (420), navigation module (800), latency monitoring module (890), calculated metrics module (460, 490) and power source (60). Scanner (100) are remote from the Cloud (570) and include X-rays, ultrasound, computerized tomography (CT), magnetic resonance imaging (MRI), dual-energy X-ray absorptiometry (DEXA) and any other state-of-the art scanner. Scanners communicate with calculated metrics module (460, 490). Communications devices (200) include desktop computers, tablet computers, smart phones or any other device, acceptable in the art, capable of encrypting, sending and receiving data. Communication devices (200) communicate with calculated metrics module (460, 490) and can also communicate with different communications devices (200). Audiovisual displays (900) communicate with communications devices (200) via audio, keystrokes and/or touch. Calculated metrics module (460, 490) directs manufacturing device (500) to manufacture patient specific reconstruction (600R). As long as there is connectability to the Internet or other rapid data delivery network and power source (60), the computerized methods of making an optimal patient-specific orthopedic reconstruction (123C), (123B) and (123B) are functional with the remote components being at different locations. By way of illustration, clinical settings (840), manufacturing device (500) and stationary and mobile communications are remote from the Cloud (570), and can also be remote from each other.

While the figures and descriptions provided herein exemplify reconstructions for major joints and bones such as the femur, humerus, and vertebrae, the computerized methods (123A, 123B, 123C) and resulting optimal reconstructions (600R) are equally applicable to all other anatomical structures requiring orthopedic or regenerative intervention. It is expressly contemplated that the target zone (40) may comprise smaller or specialized structures, including but not limited to the wrist (carpus), ankle (tarsus), fingers and toes (phalanges), and facial or cranial cartilage such as the ears (auricle) or nose. Furthermore, the fabrication of the optimal patient-specific orthopedic reconstruction (600R) may utilize the non-uniform porous lattice density map and diverse material compositions (e.g., graphene, biodegradable polymers, or hydroxyapatite) to replicate the specific mechanical and biological requirements of these micro-anatomical target zones (40). Consequently, the scope of the present invention extends to any patient-specific reconstruction (600R) generated by the optimized metric output (490, 490S) to achieve the biomechanical and prognostic goals predicted by the CPSS (123S) or OPS (123Q).

Applicant has enabled, described and disclosed the invention as required by Title 35 of the United States Code.

1. A method (123A) for manufacturing a patient-specific three-dimensional (3D) orthopedic reconstruction having a non-uniform internal structure, the method comprising:
a) receiving, via a secure encrypted communication protocol, patient-specific digitized medical scans of a target anatomical zone;
b) tagging, sending, and securely storing patient information in a non-transitory memory regarding:
i) one or more patient-specific digitized medical scans of a target zone relative to X, Y, and Z axes of the target zone; and
ii) medical history, current medical conditions, clinical observations, or medical test results;
c) executing, by a processor of a computing apparatus, a machine-learning model trained on an aggregate of historical biomechanical outcome data to:
i) transform the medical scans into a 3D digital model defining the spatial volume of the target anatomical zone;
ii) computationally simulate a plurality of biomechanical load vectors across the 3D digital model to calculate a localized stress distribution map; and
iii) dynamically modify the 3D digital model by varying a porous lattice density across its volume in response to the localized stress distribution map to minimize stress shielding; and
d) controlling a 3D manufacturing apparatus to fabricate the 3D orthopedic reconstruction based on the modified 3D digital model, wherein the 3D manufacturing apparatus simultaneously generates a longitudinal conduit through the varying porous lattice during fabrication to provide a pathway for vascularization.

2. The method (123A) of making the optimal patient-specific three-dimensional reconstruction of claim 1, wherein the optimal patient-specific three-dimensional reconstruction comprises a first section and a second section opposed to the first section and the second section is tapered such that the cross-sectional area of the optimal patient-specific three-dimensional area is less proximate the patient's target zone.

3. The method (123A) of making the optimal patient-specific three-dimensional reconstruction of claim 2, wherein the optimal metric for fabricating the optimal patient-specific three-dimensional reconstruction generates simultaneously a conduit extending through the optimal patient-specific three-dimensional reconstruction about the longitudinal axis of the optimal patient-specific three-dimensional reconstruction during a 3D printing process.

4. The method (123A) of making the optimal patient-specific three-dimensional reconstruction of claim 3, wherein the optimal patient-specific three-dimensional reconstruction comprises bulges, serrations, or wings that are computationally optimized to inhibit over-insertion of the optimal patient-specific three dimensional reconstruction, increase the surface area for bone ingrowth, dissipate loads transmitted from adjacent levels proximate the target zone, and a connector for coupling devices distinct from the optimal patient-specific three dimensional reconstruction.

5. The method (123A) of making the optimal patient-specific three-dimensional reconstruction of claim 4, wherein the computationally optimized bulges, serrations, or wings are generated according to an algorithm that dynamically modifies the geometry of the optimal patient-specific three-dimensional reconstruction in response to the optimal metric for fabricating the optimal patient-specific three-dimensional reconstruction, thereby creating a non-uniform, patient-specific structural lattice that provides a greater load-bearing capacity and a reduced risk of stress shielding.

6. A method (123B) for manufacturing an optimal patient-specific three-dimensional (3D) reconstruction for a target anatomical zone, the method comprising:
a) receiving patient-specific digitized medical scans of the target anatomical zone;
b) tagging, sending, and securely storing patient information in a non-transitory memory regarding:
i) the one or more patient-specific digitized medical scans of the target anatomical zone relative to X, Y, and Z axes of the target anatomical zone; and
ii) medical history, current medical conditions, clinical observations, or medical test results;
c) executing a software on a computing apparatus to:
i) process the medical scans and generate a patient-specific 3D template and a patient-specific 3D model of the target anatomical zone identified in a surgical plan; and
ii) calculate an Osseointegration Probability Score by comparing the 3D model with the 3D template and an aggregate comprising the surgical plan, the medical history, and a biomechanical load simulation of the 3D model; and
d) controlling a 3D manufacturing apparatus to fabricate the 3D reconstruction based on an optimal metric derived from the Osseointegration Probability Score.

7. The method (123B) of claim 6, wherein the optimal patient-specific three-dimensional reconstruction comprises one or more distinct outward sections.

8. The method (123B) of claim 7, wherein the optimal patient-specific three-dimensional reconstruction comprises at least one of bulges, serrations, or wings that are computationally optimized to inhibit over-insertion into the target anatomical zone, increase surface area for bone ingrowth, and dissipate loads transmitted from adjacent anatomical levels proximate the target anatomical zone.

9. The method (123B) of claim 8, wherein the optimal metric derived from the Osseointegration Probability Score controls the manufacturing apparatus to generate simultaneously a conduit extending through the optimal patient-specific three-dimensional reconstruction about a longitudinal axis of the optimal patient-specific three-dimensional reconstruction during a 3D printing process.

10. The method (123B) of claim 9, wherein the computationally optimized bulges, serrations, or wings are generated according to an algorithm that dynamically modifies a geometry of the optimal patient-specific three-dimensional reconstruction in response to the optimal metric, thereby creating a non-uniform, patient-specific structural lattice providing a greater load-bearing capacity and a reduced risk of stress shielding.

11. The method (123B) of claim 10, wherein the Osseointegration Probability Score is calculated by executing the software on the computing apparatus to employ a machine learning model trained on the aggregate data to predict a likelihood of successful fusion and bone ingrowth for the patient-specific three-dimensional reconstruction.

12. A method (123C) for manufacturing an optimal patient-specific three-dimensional reconstruction for a target zone in a current patient, the process compliant with jurisdictional regulatory bodies and comprising the steps of:
a) generating patient-specific digitized medical scans of the target zone using one or more medical scanners;
b) encrypting communications from the one or more medical scanners to a computing apparatus and a manufacturing apparatus, and decrypting communications received by the computing apparatus and the manufacturing apparatus;
c) tagging, sending, and securely storing patient information in a storage array of the computing apparatus, the information including the patient-specific digitized medical scans defining the spatial volume of the target zone relative to X, Y, and Z axes of the target zone, and associated medical data; d) executing a software on the computing apparatus to:
i) process the medical scans to generate a patient-specific three-dimensional model of the target zone;
ii) compare the patient-specific model with an aggregate of historical implant and outcome data from the patient and prior patients to calculate a Comprehensive Prognostic Success Score predicting the likelihood of overall longer-term success, including likelihood of successful osseointegration, infection resistance, material wear characteristics, and patient quality-of-life indications;
iii) calculate a first set of patient-specific metric outputs based on the current patient-specific three-dimensional model, a biomechanical load simulation of the patient-specific anatomical data, and the Comprehensive Prognostic Success Score;
iv) calculate a second set of patient-specific optimized metric outputs by analyzing the first set of patient-specific metric outputs in view of the aggregate historical data to improve the fit and performance of the optimal patient-specific three-dimensional reconstruction; and e) transmitting an optimal metric selected from the first set of patient-specific metric outputs, the second set of optimized metric outputs, or a combination thereof to a manufacturing apparatus for the fabrication of the patient-specific three-dimensional reconstruction.

13. The method (123C) of claim 12, wherein the optimized metric outputs comprise a non-uniform porous lattice density map, wherein the density is dynamically varied across a volume of the orthopedic reconstruction based on the biomechanical load simulation to reduce stress shielding.

14. The method (123C) of claim 13, wherein the 3D manufacturing apparatus simultaneously generates a longitudinal conduit through the orthopedic reconstruction about a longitudinal axis of the orthopedic reconstruction during a 3D printing process.

15. The method (123C) of claim 14, wherein the orthopedic reconstruction comprises at least one of bulges, serrations, or wings that are computationally optimized based on the lattice density map to increase surface area for bone ingrowth.

16. The method (123C) of claim 12, wherein the calculation of the Comprehensive Prognostic Success Score includes processing patient gait analysis data and bone mineral density readings to refine the prediction of material wear.

17. The method (123C) of claim 12, wherein the 3D orthopedic reconstruction is a spinal reconstruction comprising a tapered geometry optimized based on the Comprehensive Prognostic Success Score to match an individual anatomical alignment of the patient.

18. The method (123C) of claim 12, wherein a deep learning neural network trained on the aggregate is employed to calculate the Comprehensive Prognostic Success Score.

19. The method (123C) of claim 12, wherein the biomechanical load simulation comprises a finite element analysis model incorporating predicted muscle force vectors derived from the digitized medical scans.

20. The method (123C) of claim 12, wherein the optimized metric outputs include a selected material composition determined by the Comprehensive Prognostic Success Score to enhance infection resistance.
